(19) United States
(12) Patent Application Publication
(10) Pub. No.: US 2023/0190663 A1
(43) Pub. Date: Jun. 22, 2023

(54) PRUSSIAN BLUE NANOPARTICLES FUNCTIONALIZATION WITH LATENCY REVERSING AGENTS AND BROADLY NEUTRALIZING ANTIBODIES, AND APPLICATIONS THEREOF

(71) Applicant: THE GEORGE WASHINGTON UNIVERSITY, Washington, DC (US)

(72) Inventors: ROHAN FERNANDES, Bethesda, MD (US); PREETHI BALA BALAKRISHNAN, Arlington, VA (US); CARISSA STOVER, Silver Spring, MD (US); ALBERTO BOSQUE, Washington, DC (US); INDRA SARABIA, Washington, DC (US); ANVITHA SHRI RAMANUJAM, Fairfax, VA (US)

(21) Appl. No.: 17/950,573

(22) Filed: Sep. 22, 2022

Related U.S. Application Data
(63) Continuation of application No. PCT/US21/26632, filed on Apr. 9, 2021.
(60) Provisional application No. 63/007,588, filed on Apr. 9, 2020.

(51) Int. Cl. A61K 9/50; A61K 45/06; A61P 31/18

(52) U.S. Cl. CPC A61K 9/5073; A61K 45/06; A61P 31/18; A61K 9/5146

(57) ABSTRACT

Embodiments of the instant disclosure relate to novel compositions and methods for the treatment of retroviruses (e.g., HIV) and the reduction and/or eradication of latent HIV reservoirs. Compositions herein may include biofunctionalized nanocomposites comprised of a core nanoparticle formed of Prussian blue materials, a shell obtained by partially or completely encapsulating the Prussian blue core with at least one biocompatible coating, and at least one biomolecule attached to, or absorbed to, the biocompatible coating and uses thereof.

### CROSS-REFERENCE TO RELATED APPLICATIONS

This patent application is a continuation of PCT/US2021/026632, filed Apr. 9, 2021, for PRUSSIAN BLUE NANOPARTICLES FUNCTIONALIZATION WITH LATENCY REVERSING AGENTS AND BROADLY NEUTRALIZING ANTIBODIES, AND APPLICATIONS THEREOF, which claims the benefit of U.S. Provisional Patent Application Ser. No. 63/007,588, filed on Apr. 9, 2020, each of which is incorporated herein by reference in its entirety.

### GOVERNMENTAL RIGHTS

This invention was made with government support under R01AI124722 awarded by the National Institutes of Health and the National Institute of Allergy and Infectious Diseases. The government has certain rights in the invention.

### FIELD

The present disclosure generally relates to compositions and methods for the treatment of retroviruses (e.g., HIV) and the reduction and/or eradication of latent HIV reservoirs and methods of use thereof.

### BACKGROUND

Highly active antiretroviral therapy (HAART) serves as an effective strategy to combat HIV infections by suppressing viral replication in patients with HIV/AIDS. However, HAART does not provide HIV/AIDS patients with a sterilizing or functional cure, and introduces several deleterious comorbidities. Moreover, the virus is able to persist within latent reservoirs, both undetected by the immune system and unaffected by HAART, increasing the risk of a viral rebound. Elimination of the HIV-1 latent reservoir is critical to achieving HIV-1 eradication in vivo. One approach to eliminating the viral reservoir is the “shock and kill” strategy. In this approach, drugs known as latency reversing agents (LRAs), are administered to reverse HIV-1 latency and induce viral production, ultimately resulting in the death of infected cells by direct viral cytopathic effects or immune-mediated clearance. Unfortunately, LRAs currently available for clinical use fail to significantly reduce reservoir size and exert weak effects on HIV-1 transcription and reactivation. As such, there is a need in the art for improved compostions and methods of use thereof for the elimination of the HIV-1 latent reservoir and treatment of HIV/AIDS.

### SUMMARY

The present disclosure provides for compositions and methods for the treatment of retroviruses (e.g., HIV) and the reduction and/or eradication of latent HIV reservoirs. Compositions herein may include biofunctionalized nanocomposites comprised of a core nanoparticle formed of Prussian blue materials, a shell obtained by partially or completely encapsulating the Prussian blue core with at least one biocompatible coating, and at least one biomolecule attached to, or absorbed to, the biocompatible coating. In embodiments, biofunctionalized nanocomposites herein can be comprised of Prussian blue materials. In some embodiments, Prussian blue materials herein may be iron hexacyanoferrate (II) compounds. In some embodiments, Prussian blue materials herein may be represented by general formula (I):

AₓB_{w}M₄[M′(CN)₆]_{z}·nH₂O (I),

wherein, x is from 0, 0.1 or about 0.1 to about 1; w is from 0, 0.1 or about 0.1 to about 1; z is from about 0.1 to about 4; n is from about 0.1 to about 24; A represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof; B represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof, M represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof; and M′ represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof.

In some embodiments, biofunctionalized nanocomposites herein may have least one biocompatible coating wherein the coating may comprise citrate, dextran, chitosan, silica, polyethylene glycol (PEG), avidin; a protein, a nucleic acid, a carbohydrate, a lipid, neutravidin, streptavidin, gelatin, collagen, fibronectin, albumin, a serum protein, a lysozyme, a phospholipid, a polyvinyl pyrrolidone (PVP), a polyvinyl alcohol, polyethylene glycol diacrylate, polyethylenimine (PEI), or any combination thereof. In some embodiments, at least one biocompatible coating herein may be citrate, polyethylenimine (PEI), or any combination thereof. In some embodiments, biofunctionalized nanocomposites herein may have least two biocompatible coatings. In some embodiments, biofunctionalized nanocomposites herein may have at least one coating layer of citrate, at least one coating layer of PEI, or any combination thereof.

In some embodiments, biofunctionalized nanocomposites herein may have at least one biomolecule attached to, or absorbed to, the biocompatible coating. In some embodiments, biomolecules that are attached to, or absorbed to, the biocompatible coating of biofunctionalized nanocomposites herein may be an antibody, a peptide, a protein, an enzyme, an amino acid, a nucleic acid, a carbohydrate, a fat, an aptamer, a small molecule, a synthetic molecule, or any combination thereof.

In some embodiments, biomolecules herein that are attached to, or absorbed to, the biocompatible coating of biofunctionalized nanocomposites herein may be at least one latency reversing agent. In some examples, latency reversing agents attached to, or absorbed to, the biocompatible coating of biofunctionalized nanocomposites herein may be prostratin, bryostratin, ingenol, TNF-alpha, IL-15, IL-15 superagonist or any combination thereof. In some examples, latency reversing agents herein can be at least one pathogen recognition receptor agonist. In some aspects, a pathogen recognition receptor agonist for use in biofunctionalized nanocomposites herein may be toll-like receptor activators, RIG-1-like receptors activators, cytosolic DNA sensors activators, cyclic dinucleotides, or any combination thereof. In some examples, latency reversing agents for use in biofunctionalized nanocomposites herein can be at least one toll-like receptor (TLR) agonist. In some examples, a TLR agonist for use herein may be a TLR-2 agonist, a TLR-3 agonist, a TLR-4 agonist, a TLR-5 agonist, a TLR-7 agonist, a TLR-7/8 agonist, a TLR-9 agonist, or any combination thereof. In some aspects, latency reversing agents for use in biofunctionalized nanocomposites herein may be a high molecular weight polyinosine-polycytidylic acid (poly I:C), low molecular weight poly I:C, Flagellin, GS-9620, R-848, CpG-ODNs, 5′ppp-dsRNA, 3p-hpRNA, Poly(I:C)/LyoVec complexes, Poly(dA:dT)/LyoVec complexes, 2′3′-cGAMP, 3′3′-cGAMP, c-di-AMP, c-di-GMP, cAIMP (CL592), cAIMP Difluor (CL614), cAIM(PS)2 Difluor (Rp/Sp) (CL656), 2′2′-cGAMP, 2′3′-cGAM(PS)2 (Rp/Sp), 3′3′-cGAMP Fluorinated, c-di-AMP Fluorinated, 2′3′-c-di-AMP, 2′3′-c-di-AM(PS)2 (Rp,Rp), c-di-GMP Fluorinated, 2′3′-c-di-GMP, c-di-IMP, dsDNA-EC, G3-YSD, HSV-60, ISD, ODN TTAGGG (A151), Poly(dA:dT), Poly(dG:dC), VACV-70, or any combination thereof. In some embodiments, biofunctionalized nanocomposites herein may have at least one latency reversing agent attached to, or absorbed to, the biocompatible coating wherein the at least one latency reversing agent can be a poly I:C (e.g., a high molecular weight poly I:C, a low molecular weight poly I:C, or any combination thereof).

In some embodiments, biomolecules herein attached to, or absorbed to, the biocompatible coating of the disclosed biofunctionalized nanocomposites may be at least one broadly neutralizing antibody (bnAB) against HIV. In some examples, bnABs attached to, or absorbed to, the biocompatible coating of biofunctionalized nanocomposites herein may be 3BNC117, VRC01, VRC02, 10-1074, or any combination thereof.

In some embodiments, biofunctionalized nanocomposites herein may have at least one biocompatible coating comprising a positively charged coating. In some examples, biofunctionalized nanocomposites herein may have a positively charged coating that can bind to at least one negatively charged biomolecule. In some examples, negatively charged biomolecules that may be bound to a positively charged coating of biofunctionalized nanocomposites herein may be at least one latency reversing agent, at least one negatively charged bnAb, or any combination thereof.

Certain aspects of the present disclosure provide for methods of treating HIV. In some embodiments, methods herein of treating HIV in a subject in need thereof can comprise administering to the subject any one of the biofunctionalized nanocomposites herein wherein the subject in need thereof is a human having or suspected of having HIV.

In some embodiments, methods herein may further include administering at least one latency reversing agent separately from any latency reversing agent contained within a biofunctionalized nanocomposite herein. In some embodiments, methods herein may further include administering at least one bnAb separately from any bnAb contained within a biofunctionalized nanocomposite herein. In some embodiments, methods herein may include administration of a latency reversing agent more than once. In some embodiments, methods herein may include administration of a bnAb more than once.

In some embodiments, methods herein may further include administering at least one anti-retroviral therapeutic. In some examples, methods of further administering at least one anti-retroviral therapeutic as disclosed herein may include administering enfuvirtide, zidovudine, abacavir, lamivudine, emtricitabine, tenfovir, nevirpine, efavirenz, etravirine, rilpivirine, raltegravir, elvitegravir, dolutegravir, lopinavir, indinavir, nelfinavir, amprenavir, ritonavir, darunavir, atazanavir, bevirimat, vivecon, stavudine, didanosine, delavirdine, nevirapine, fosamprenavir, saquinavir, tipranavir, maraviroc, or any combination thereof. In some examples, methods of further administering at least one anti-retroviral therapeutic as disclosed herein may include administering at least one nucleoside/nucleotide reverse transcriptase inhibitor (NRTI), at least one non-nucleoside reverse transcriptase inhibitor (NNRTI), at least one protease inhibitor, at least one fusion or entry inhibitor, at least one integrase inhibitor, or any combination thereof.

Certain aspects of the present disclosure provide for pharmaceutical compositions comprising any one of the biofunctionalized nanocomposites disclosed herein. In some embodiments, pharmaceutical compositions herein may have any one of the biofunctionalized nanocomposites disclosed and at least one pharmaceutically acceptable excipient.

In some embodiments, methods herein may reduce and/or eradicate latent HIV reservoirs in a cell of a subject. In some embodiments, methods herein may comprise delivering any one of the biofunctionalized nanocomposites and/or any one of the pharmaceutical compositions disclosed herein to a cell having a latent reservoir of HIV virus.

In some embodiments, methods herein may be administered to a subject. In some aspects, methods herein may be applied to subjects wherein the subject is a human subject. In some aspects, methods herein may be applied to human subjects wherein the human subject may have or may be suspected of having HIV.

In certain embodiments, kits are provided herein. In certain embodiments, compositions of the instant disclosure can be included in the kit, together or in separate containers. In certain embodiments, kits are provided for the practice of any one of the methods disclosed herein. In some embodiments, kits can include one or more of the biofunctionalized nanocomposites disclosed herein, one or more latency reversing agents, one or more bnABs, one or more anti-retroviral therapeutics, or a combination thereof.

### BRIEF DESCRIPTION OF THE DRAWINGS

The description will be more fully understood with reference to the following figures and data graphs, which are presented as variations of the disclosure and should not be construed as a complete recitation of the scope of the disclosure, wherein:

FIG. 1 illustrates a schematic depicting a method of coating Prussian blue nanoparticles in accordance with embodiments of the present disclosure.

FIGS. 2A-2E illustrate the characterization of Prussian blue nanoparticles in accordance with embodiments of the present disclosure. FIG. 2A shows a graph depicting measurement of nanoparticle size. FIG. 2B shows a graph depicting zeta potential of the nanoparticles. FIG. 2C shows a graph depicting absorption peaks measured for the nanoparticles. FIG. 2D shows a graph depicting the collection yield of the nanoparticles after each coating step. FIG. 2E shows a graph depicting the percentage of poly I:C attached to a nanoparticle after coating.

FIGS. 3A and 3B illustrate latency reversal in model of latent HIV cells after treatment with nanoparticles in accordance with embodiments of the present disclosure. FIG. 3A shows a graph depicting the percentage of GFP expression in J-LAT cells after the cells were either transfected with Citrate-PBNPs, PEI-PBNPs, polyIC-PBNPs, and free poly I:C (transfected) or Citrate-PBNPs, PEI-PBNPs, polyIC-PBNPs, and free poly I:C were added to the cell culture medium (untransfected). FIG. 3B shows a graph depicting the percentage of viable J-LAT cells remaining after the cells were either transfected with Citrate-PBNPs, PEI-PBNPs, polyIC-PBNPs, and free poly I:C (transfected) or Citrate-PBNPs, PEI-PBNPs, polyIC-PBNPs, and free poly I:C were added to the cell culture medium (untransfected).

FIGS. 4A-4D illustrate latency reversal of the HIV virus by polyIC-PBNPs and free poly I:C in accordance with embodiments of the present disclosure. FIG. 4A shows a graph depicting the percentage of GFP expression in J-LAT cells after the cells were treated with increasing concentrations of polyIC-PBNPs and free poly I:C where the polyIC-PBNPs and free poly I:C were added to the cell culture medium (untransfected). FIG. 4B shows a graph depicting the percentage of viable J-LAT cells after the cells were treated with increasing concentrations of polyIC-PBNPs and free poly I:C where the polyIC-PBNPs and free poly I:C were added to the cell culture medium (untransfected). FIG. 4C shows a graph depicting the percentage of GFP expression in F2 cells lacking MAVS after the cells were treated with increasing concentrations of polyIC-PBNPs and free poly I:C where the polyIC-PBNPs and free poly I:C were added to the cell culture medium (untransfected). FIG. 4D shows a graph depicting the percentage of viable F2 cells lacking MAVS after the cells were treated with increasing concentrations of polyIC-PBNPs and free poly I:C where the polyIC-PBNPs and free poly I:C were added to the cell culture medium (untransfected).

FIG. 5 illustrates the effective concentration of polyIC-PBNPs and free poly I:C on viral reactivation in accordance with embodiments of the present disclosure. Increasing concentrations of polyIC-PBNPs and free poly I:C were added to the cell culture medium of J-LAT cells overnight before the percentage of GFP expression was assessed.

FIGS. 6A-6G illustrate immunological responses to polyIC-PBNPs and free poly I:C in healthy human peripheral blood mononuclear cells (PBMCs) in accordance with embodiments of the present disclosure. FIG. 6A shows a graph depicting the percentage of live PBMCs following an overnight incubation of the cells with polyIC-PBNPs and free poly I:C. FIG. 6B shows a graph depicting the ratio of T cells (CD3+ cells) to other mononuclear cells (CD3− cells) in PBMCs following an overnight incubation of the cells with polyIC-PBNPs and free poly I:C. FIG. 6C shows a graph depicting the ratio of T helper cells (CD4+ cells) and cytotoxic T cells (CD8+ cells) in PBMCs following an overnight incubation of the cells with polyIC-PBNPs and free poly I:C. FIG. 6D shows a graph depicting the percentage of T helper cells (CD4+ cells) that expressed the immune cell activation marker (CD69) in PBMCs following an overnight incubation of the cells with polyIC-PBNPs and free poly I:C. FIG. 6E shows a graph depicting the percentage of cytotoxic T cells (CD8+ cells) that expressed the immune cell activation marker (CD69) in PBMCs following an overnight incubation of the cells with polyIC-PBNPs and free poly I:C. FIG. 6F shows a graph depicting the percentage of NK cells (CD56+ cells) in PBMCs following an overnight incubation of the cells with polyIC-PBNPs and free poly I:C. FIG. 6G shows a graph depicting the percentage of activated NK cells (CD56+CD69+ cells) in PBMCs following an overnight incubation of the cells with polyIC-PBNPs and free poly I:C.

FIG. 7 illustrates viral reactivation in J-LAT cells lacking MAVS by treatment with a supernatant of PBMCs incubated overnight with varying concentrations of PolyIC-PBNP or Free Poly I:C in accordance with embodiments of the present disclosure.

### DETAILED DESCRIPTION

In the following sections, certain exemplary compositions and methods are described in order to detail certain embodiments of the invention. The disclosure may be understood by reference to the following detailed description, taken in conjunction with the drawings as described below. It will be obvious to one skilled in the art that practicing the certain embodiments does not require the employment of all or even some of the specific details outlined herein, but rather that concentrations, times and other specific details can be modified through routine experimentation. In some cases, well known methods, or components have not been included in the description.

Embodiments of the instant disclosure relate to novel methods and compositions for the treatment of retroviruses (e.g., HIV) and for the reduction and/or eradication of latent HIV reservoirs. In some embodiments, compositions herein may comprise at least one biofunctionalized nanocomposite. In some embodiments, methods herein may treat HIV in a subject, the method comprising administering a least one of biofunctionalized nanocomposite disclosed herein to the subject.

### I. Compositions

In certain embodiments, the present disclosure provides compositions containing at least one biofunctionalized nanocomposite. A composition disclosed herein may encompass a nanoparticle formed of Prussian blue materials, a biocompatible coating, and a biomolecule.

(a) Prussian Blue Nanoparticles

In some embodiments, compositions disclosed herein may comprise a nanoparticle formed of at least one or more Prussian blue materials. As used herein, “Prussian blue materials”, “Prussian blue” and “Prussian blue compounds” are used interchangeably. The compositions disclosed herein may include Prussian blue materials, such as Prussian blue nanoparticles. The Prussian blue nanoparticles may be functionalized with materials that serve as latency reversal agents and/or antibodies for HIV. Historically, Prussian blue material was employed as a pigment and first synthesized in the 18^{th} century. The Prussian blue materials are made up of a coordination polymer involving a network of metal ions linked with a nitrogen containing organic bridge. In particular, the base structure of Prussian blue materials include a network of iron ions which are coordinated with a plurality of cyanide bridges.

Prussian blue materials generally include iron (II, III) hexacyanoferrate (II, III) as a chemical structure. This base structure may be modified with attachment, by covalent or non-covalent mans, with various other metals, elements or functional groups.

The core chemical structure Prussian blue materials include ions of Fe and the coordination complex [Fe(CN)₆] and which also include hydrate forms. The Prussian blue materials have forms which may be soluble or insoluble in water. The most common of these Prussian blue materials include the chemical formula KFe^{III}[Fe^{III}(CN)₆] and Fe^{III}₄[Fe^{II}(CN)₆]₃, and which may be employed herein. Prussian blue materials also include the FDA-approved material that is marketed as Radiogardase® by Heyltex. The Radiogardase Prussian blue materials may be the insoluble form and have the formula Fe^{III}₄[Fe^{II}(CN)₆]₃·nH₂O where n is some integer.

The Prussian blue materials may be salts of the coordination complex [Fe(CN)₆], wherein such salts may include for instance Li, Na, K, Rb, Cs, NH₄ or Tl and which may include hydrate forms. Rather than Fe, other metals may be employed in the coordination complex.

In various aspects as disclosed herein, the Prussian blue materials may be represented by general formula (I):

AₓB_{w}M₄[M′(CN)₆]_{z}·nH₂O (I),

wherein
x is from 0, 0.1 or about 0.1 to about 1;
w is from 0, 0.1 or about 0.1 to about 1;
z is from about 0.1 to about 4; and
n is from about 0.1 to about 24.
A represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof,
B represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof,
M represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof,
M′ represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof.

Any combination of the aforementioned elements for A, B, M, and M′, independently of one another, may be employed, and wherein a particular element for M and M′ is Fe.

The Prussian blue materials for use according to the disclosure herein may be represented by general formula (II):

AₓFe_{y}^{III}[Fe^{II}(CN)₆]_{z}·nH₂O (II),

wherein

A represents at least one of Li, Na, K, Rb, Cs, NH₄ or Tl, in any oxidation state;

x is from 0 to about 1;
y is from about 0.1 to about 4;
z is from about 0.1 to about 4; and
n is from about 1 to about 24

The Prussian blue materials for use according to the disclosure herein may be represented by general formula (III):

Fe₄^{III}[Fe^{II}(CN)₆]₃·nH₂O (III)

wherein n is from 1 to about 24

The Prussian blue materials for use according to the disclosure herein may comprise the chemical formula (IV):

Fe^{III}₄[Fe^{II}(CN)₆]₃·nH₂O (IV).

The Prussian blue materials for use according to the disclosure herein may comprises potassium ferrocyanide, which may have the chemical formula (V);

KFe^{III}[Fe^{II}(CN)₆] (V),

The Prussian blue materials can be formed in a solvent in which the reaction between the metallic salt and the metallic cyanide described above occurs is an organic solvent. In an aspect, the organic solvent can be hydrophilic to any degree or hydrophobic to any degree. In a preferred aspect, the organic solvent comprises, consists essentially of, or consists of hexane; benzene; toluene; diethyl ether; chloroform; 1,4-dioxane; ethyl acetate; tetrahydrofuran (THF); dichloromethane; acetone; acetonitrile (MeCN); dimethylformamide (DMF); dimethyl sulfoxide (DMSO); a polar protic solvent; acetic acid; n-butanol; isopropanol; n-propanol; ethanol; methanol; formic acid; and any combination thereof, so long as the metallic salt and the metallic cyanide are sufficiently dissolved in the combination and the reaction proceeds in this combination of solvents.

The Prussian blue materials may be synthesized by reacting the selected the metals above as a salt with the selected metal cyanide ([M′(CN)₆]³⁻) in a solvent. The metallic salt may include any of the aforementioned metals in a metallic salt of a chloride, a nitrate, a nitrite, a sulfate, a fluorinate, a glutamate, an acetate, a carbonate, a citrate, a phosphate, a sulfate and any combination thereof. The solvent in which the reaction between the metallic salt and the metallic cyanide described above occurs may not be particularly limited, so long as the reaction proceeds in this solvent. In an aspect, the solvent comprises, consists essentially of, or consists of water, air, or an organic solvent.

The solvent in which the reaction between the metallic salt and the metallic cyanide described above occurs is an organic solvent. In an aspect, the organic solvent can be hydrophilic to any degree or hydrophobic to any degree. In a preferred aspect, the organic solvent comprises, consists essentially of, or consists of hexane; benzene; toluene; diethyl ether; chloroform; 1,4-dioxane; ethyl acetate; tetrahydrofuran (THF); dichloromethane; acetone; acetonitrile (MeCN); dimethylformamide (DMF); dimethyl sulfoxide (DMSO); a polar protic solvent; acetic acid; n-butanol; isopropanol; n-propanol; ethanol; methanol; formic acid; and any combination thereof, so long as the metallic salt and the metallic cyanide are sufficiently dissolved in the combination and the reaction proceeds in this combination of solvents.

The aforementioned composition can be synthesized in the form of colloidal nanoparticles. The Prussian blue nanoparticles (PBNPs) may range in sizes of from about 1 nanometer (nm) to about 1 micron (m), alternatively from 2 nm to about 1 (m), alternatively from 5 nm to about 500 nm, alternatively from about 10 nm to about 300 nm, including combinations of the aforementioned end points. Furthermore, these PBNPs may exhibit a broad absorbance peak that can range from, for instance the wavelength may range from about 600 to about 1200 nm, alternatively from about 650 to about 950 nm, including combinations of the aforementioned end points.

(b) Biocompatible Coatings

In certain embodiments, compositions disclosed herein may comprise a shell partially or completely encapsulating a nanoparticle. In some aspects, a shell encapsulates about 25%, about 50%, about 75%, or about 100% of the nanoparticle. In a preferred aspect, a shell completely encapsulates a nanoparticle formed of Prussian blue materials as disclosed herein.

In various embodiments, a shell is comprised of a biocompatible coating. In an a aspect, a biocompatible coating comprises one or more biocompatible materials assisting to in vivo and in vitro use of compositions disclose herein. In some embodiments, a biocompatible coating of the shell may comprise at least one material selected from the group consisting of citrate; dextran; chitosan; silica; polyethylene glycol (PEG); avidin; a protein; a nucleic acid; a carbohydrate; a lipid; neutravidin; streptavidin; gelatin; collagen; fibronectin; albumin; a serum protein; lysozyme; a phospholipid; a polyvinyl pyrrolidone (PVP); a polyvinyl alcohol; a polyethylene glycol diacrylate; polyethylenimine (PEI); and a combination thereof. Without wishing to be bound to any particular theory, the biocompatible coating is believed to prevent the compositions from aggregating and to prevent leakage of ions from the core to the surrounding environment.

In some embodiments, a dextran of the biocompatible coating may comprise a dextran that is a complex, branched polysaccharide having chains of varying lengths, preferably chains having lengths of from about 3 to about 2000 kDa. In other embodiments, a chitosan of the biocompatible coating may comprise a linear polysaccharide having randomly distributed units of β-(1-4)-linked D-glucosamine (deacetylated unit) and units of N-acetyl-D-glucosamine (acetylated unit). In still other embodiments, a silica of the biocompatible coating may comprise an oxide of silicon with the chemical formula SiO₂. In yet other embodiments, a polyethylene glycol (PEG) of the biocompatible coating may comprise polyethylene oxide (PEO) or polyoxyethylene oxide (POE). In other embodiments, an avidin of the biocompatible coating may comprise a protein produced in the oviducts of birds, reptiles and amphibians deposited in the whites of their eggs. In yet other embodiments, an albumin of the biocompatible coating may comprise bovine serum albumin (BSA, fraction V), human serum albumin (HSA) and all serum albumin derived from mammals. In an aspect, serum proteins of the biocompatible coating may comprise at least one member selected from the group consisting of Orosomucoid; antitrypsin; alpha-1 antichymotrypsin; alpha-2 macroglobulin (AMG); haptoglobin; transferrin; beta lipoprotein (LDL); immunoglobulin A (IgA); immunoglobulin M (IgM); immunoglobulin G (IgG); immunoglobulin E (IgE); and immunoglobulin D (IgD). In some embodiments, a lysozyme of the biocompatible coating may be of N-acetylmuramide glycanhydrolase. In still other embodiments, phospholipids of the biocompatible coating may comprise of all natural phospholipids and synthetic phospholipids. Non-limiting examples of natural phospholipids and synthetic phospholipids include DMPA, DPPA, DSPA DDPC, DLPC, DMPC, DPPC, DSPC, DOPC, POPC, DEPC DMPG, DPPG, DSPG, POPG DMPE, DPPE, DSPE DOPE DOPS mPEG-phospholipid, polyglycerin-phospholipid, functionalized-phospholipid, and terminal activated-phospholipid. In other embodiments, a polyvinyl pyrrolidone (PVP) of the biocompatible coating may comprise a polymer made from repeating monomer N-vinylpyrrolidone units. In an aspect, the molecular weight of the PVP is not particularly limited, as long as the PVP is suitable for use in the biocompatible coating of the present disclosure. As used herein, the terms “polyvidone” and “povidone” are synonymous with PVP. In yet other embodiments, a polyvinyl alcohol of the biocompatible coating may comprise PVOH, PVA, and PVAI. In an aspect, molecular weights of the PVOH, PVA, and PVAI are not particularly limited, as long as the PVOH, PVA, and PVAI are suitable for use in the biocompatible coating of the present disclosure. In other embodiments, a polyethylene glycol diacrylate of the biocompatible coating may comprise a polyethylene glycol terminated with acrylate groups. In an aspect, molecular weight of the polyethylene glycol diacrylate is not particularly limited, as long as the polyethylene glycol diacrylate is suitable for use in the biocompatible coating of the present disclosure. In some other embodiments, lipids of the biocompatible coating may comprise sterols, fats, oils, waxes, vitamin A, vitamin D, vitamin E, vitamin K, phospholipids of claim 5q, (mono-, di-, tri-) glycerides, or a combination thereof.

In some embodiments, a biocompatible coating of the shell of compositions disclosed herein may comprise one or more polymers. In an aspect, a polymer suitable for use in a biocompatible coating disclosed herein may be polyethylene glycol, polypropylene glycol, polyoxyethylene ether, polyanethol sulfonic acid, polyethylene imine, polymaleimide, polyvinyl alcohol, polyvinyl chloride, polyvinyl acetate, polyvinyl pyrrolidone, polyvinyl sulfate, polyacrylic acid, polymethacrylic acid, polylactide, polylactide glycide, or a combination thereof. In a preferred aspect, the biocompatible coating comprises polyethyleneimine (PEI).

In various embodiments, the biocompatible coating can be applied to the core of the compositions disclosed herein by a variety of physical and chemical interactions including, but not limited to: electrostatic (charge-based), covalent, hydrophobic and van der Waal's interactions. In a preferred embodiment, the biocompatible coating is applied by suspending the core in a solution comprised of one or more materials selected from the group consisting of citrate; dextran; chitosan; silica; polyethylene glycol (PEG); avidin; a protein; a nucleic acid; a carbohydrate; a lipid; neutravidin; streptavidin; gelatin; collagen; fibronectin; albumin; a serum protein; lysozyme; a phospholipid; a polyvinyl pyrrolidone (PVP); a polyvinyl alcohol; a polyethylene glycol diacrylate; polyethylenimine (PEI); and a combination thereof.

In some embodiments, a nanoparticle to be coated herein may have at least one citrate coating. In some embodiments, a nanoparticle to be coated herein may have at least one PEI coating. In some embodiments, a nanoparticle to be coated herein may have at least one citrate coating and at least one PEI coating. In some embodiments, a nanoparticle to be coated herein may have a first citrate coating and a second PEI coating.

In some embodiments, a nanoparticle to be coated herein may be a Prussian blue nanoparticle (PBNPs). In some embodiments, PBNPs may be may be provided with a first coat (also referred to as a “first layer”) which may be negatively charged. The first coat may comprise a negatively charged compound or one that may become negatively charged. The first coat may serve as a chelating agent for attachment of one or more components disclosed herein which may act as ligands. The first coat may include a compound having a plurality of chelating moieties, such as chelating acid moieties. In particular, such chelating compounds may have a plurality of carboxylic acid functional groups or their corresponding ester or salt derivatives, including polycarboxylic acids, such as two or more carboxylic acid functional groups, including two (di-carboxylic acids), three (tri-carboxylic acids), four (tetra-carboxylic acids), five (penta-carboxylic acids), six (hexa-carboxylic acids) or more carboxylic acid functional groups.

In some embodiments, the compounds herein may have a base hydrocarbon chain to which the carboxylic acids or other chelating moieties are attached including one, two, three, four, five, six, seven, eight, nine or ten or more carbons, or may have for instance, 1 to 20 carbons, or 1 to 10 carbons, or 1 to 5 carbons, or 1 to 2 carbons, which may be straight, branched, cyclic, or aromatic and where one, two, three, four or more carbons may be replaced with a nitrogen (such that the chain may include one, two, three, or more nitrogens), or may be replaced by where one, two, three, four or more carbons may be replaced with a oxygen (such that the chain may include one, two, three, or more oxygens). The carboxylic acid functional groups include their corresponding ester forms as well. Example compounds suitable for use according to the present application include having two or more include citric acid (and the corresponding ester derivative citrate), N-(hydroxyethyl) ethylenediaminetriacetic acid, 1,4,7,10-tetraazacyclotetradecane-1,4,7,10-tetraacetic acid (DOTA), diethylenetriamine pentaacetic acid (DTPA), Ethylenediaminetetraacetic acid (EDTA), ethylene glycol tetraacetic acid (EGTA), diethylene triamine pentaacetic acid, and their ester or salt derivatives.

In some embodiments, a first coating herein may have any thickness, but may be less than about 200 nm, or less than about 150 nm, or less than about 100 nm, or less than about 50 nm, or less than about 25 nm, or may range from about 5 to about 200 nm, or from about 10 to about 150 nm, or from about 25 to about 100 nm, or from about 30 to about 80 nm, or from about 40 about 50 nm.

In some embodiments, PBNPs herein may be may be provided with a second coat (also referred to as a “second layer”) which may be positively charged. The addition of the second positively charged coat can assist in the attachment of components. For instance, components may be negatively charged, or may have polarity which responds such that attachment is enhanced by the positive charge of the second coat. The second coat may comprise a positively charged compound or one that may become positively charged. The second coat can include any compound that assists attachment, covalently or non-covalently, of a biomolecule to a PBNP. The biomolecule may associate with a surface charge of the coating, wherein a cationic surface charge may interact with the negative charged components.

In some preferred embodiments, the PBNPs herein may be coated to facilitate the attachment of additional components such as latency reducing agents (LRAs), broadly neutralizing antibodies (bnAbs), or any combination thereof. In accordance with embodiments herein, such coatings may be a one coating, two coatings, or a plurality of coats. The coatings may be the same or different, or may alternate. Each coating may be made up of one or a plurality of layers of the same material. Each coating may be made up of one or a plurality of layers of different materials. In some aspects, coatings herein may be suitably adjusted so as to attach, coat or otherwise associate, with additional components such LRAs and/or bnAbs. Each of the coatings herein may be non-toxic and safe in a physiological environment.

(c) Biomolecules

In certain embodiments, compositions disclosed herein may comprise a shell partially or completely encapsulating a nanoparticle with one or more biocompatible coatings wherein at least one biomolecule may be attached to, or absorbed to, at least one biocompatible coating. In a preferred aspect, the shell completely encapsulates a nanoparticle formed of Prussian blue materials with at least one biocompatible coating(s) wherein at least one biomolecule may be attached to, or absorbed to, the biocompatible coating(s).

In various embodiments, a biocompatible coating disclosed herein may absorb at least 25%, at least 50%, or at least 75% biomolecule weight by total weight of the biocompatible coating. In other embodiments, at least 25%, at least 50%, at least 75%, at least 100% of the outer surface of the biocompatible coating has biomolecules attached.

In various embodiments, a biomolecule attached to, or absorbed to, the biocompatible coating may comprise an antibody, a peptide, a protein, an enzyme, an amino acid, a nucleic acid, a carbohydrate, a fat, an aptamer, a small molecule, a synthetic molecule or a combination thereof.

In some embodiments, at least one of the biomolecules is a nucleic acid. In some embodiments, a nucleic acid may be DNA (deoxyribonucleic acid), RNA (ribonucleic acid), a peptide nucleic acid, a morpholino-nucleic acid, a locked nucleic acid, a glycol nucleic acid, a threose nucleic acid, an oligonucleotide, or a combination thereof.

In some embodiments, the biomolecule may be an oligonucleotide. In some aspects, an oligonucleotide may be at least 5, at least 10, at least 25, at least 50, at least 75, at least 100, at least 250, or at least 500 base pairs (bp). In other aspects, an oligonucleotide may be an oligodeoxynucleotide.

In some embodiments, at least one of the biomolecules may be an antibody. As used herein, an “antibody” or “antibody molecule” is any immunoglobulin, including antibodies and fragments thereof, that binds to a specific antigen. The term includes polyclonal, monoclonal, chimeric, single domain (Dab) and bispecific antibodies. As used herein, antibody or antibody molecule contemplates recombinantly generated intact immunoglobulin molecules and immunologically active portions of an immunoglobulin molecule such as, without limitation: Fab, Fab′, F(ab′)₂, F(v), scFv, scFv2, scFv-Fc, minibody, diabody, tetrabody, single variable domain (e.g., variable heavy domain, variable light domain), bispecific, and peptabodies.

In some embodiments, compositions herein may comprise at least one antibody. In some embodiments, compositions herein may comprise at least one broadly neutralizing antibody (bnAb). Broadly neutralizing antibodies (bnAbs) are anti-HIV antibodies which can bind to the HIV virus and prevent infection. Non-liming examples of bnAbs suitable for use herein include 3BNC117, 10-1074, VRC01, VRC09, PGT121, and PG9.

In some embodiments, at least one of the biomolecules may be a peptide. In some embodiments, a peptide may consist of any sequence of 50 amino acids or less, excluding zero. In some embodiments, a peptide may consist of any sequence of about 2 amino acids to about 50 amino acids. In a preferred aspect, a peptide may consist of any sequence of 20 amino acids or less, excluding zero.

In various embodiments, a biomolecule attached to, or absorbed to, the biocompatible coating may comprise a cell. In some embodiments, cells to be attached to the nanoparticles herein can be one or more immune cells. As used herein an “immune cell” refers to a cell of the immune system. Immune cells can be categorized as lymphocytes, neutrophils, granulocytes, mast cells, monocytes/macrophages, and dendritic cells. In some aspects, immune cells for use in the compositions disclosed herein can include at least one lymphocyte. In some aspects, lymphocytes can be T-cells (CD4 T cells and/or CD8 T cells), B-cells, and natural killer (NK) cells are categorized as lymphocytes. In other aspects, an immune cell for use in the compositions disclosed herein can be a cytotoxic lymphocyte. As used herein, a “cytotoxic lymphocyte” refers to a lymphocyte capable cytolysis. For example, but not limited to, a cytotoxic lymphocyte can be capable of killing cancer cells, cells that are infected (particularly with viruses), and cells that are damaged in one or more other ways. In some aspects, a cytotoxic lymphocyte can be a NK cell or a CD8 T cell.

In some embodiments, at least one of the biomolecules may be a small molecule. “Small molecules” as used herein can refer to chemicals, compounds, drugs, and the like. In some aspects, compounds which may be employed herein are those which may have moieties which may form a positive charge in a physiological environment and/or pH. In particular, suitable compounds include those having one or a plurality of protonatable nitrogens to form an ammonium cation. Exemplary compounds include compounds with amine functional groups, in particular a plurality of protonatable secondary or tertiary amines. For instance, polycations such as polymers having a plurality of nitrogen atoms in the main chain and/or branches of the polymer, and/or amino functional groups extending from the polymer chain. In some embodiments, compounds for use herein can be any one of the compounds known in the art for the treatment of HIV. In some embodiments, compounds for use herein can be any one of the compounds known in the art for the reduction and or eradication of latent HIV reservoirs. In some embodiments, compounds for use herein can be latency reversal agents (LRAs).

(i) Latency Reversal Agents and/or Broadly Neutralizing Antibodies

After precreation of the coated PBNPs, additional therapeutic components may be attached to the surface of the PBNPs. These therapeutic components include for example LRAs as well as bnAbs. The attachment of the therapeutic components to the coated PBNPs may depend on its charge, for example whether positively or negatively charged. When the therapeutic component is negatively charged, the PBNP may have an outer positive coating, and alternatively, when the therapeutic component is positively charged, the PBNP may have a negative charge. Accordingly, the therapeutic components may have a charge opposite that of the outer coating of the PBNP.

These therapeutic components are attached onto the surface of the PBNPs through a variety of bioconjugation strategies that can include, but are not limited to: electrostatic binding, ionic bonding, hydrogen bonding, layer-by-layer assembly, adsorption, covalent bonds, van der Waals interactions, and hydrophobic interactions, or a combination of these.

The attached components may include latency reversal agents. As previously described HIV may remain latent in a subject, namely, it remains hidden and inactive inside cells of the immune system for a period of time, such as days, weeks, months or years. During this latency period a subject's immune system cannot detect and kill the virus. Latency reversal agents are those agents which reactivate the latent virus, for example by inducing HIV transcription, and thereby cause latency reversal.

LRAs as disclosed herein include DNA and/or RNA, and in particular DNA and/or RNA which would cause an immunologic response or detection from a subject's body or a cell, such as a foreign DNA and/or RNA or fragment thereof. Additionally, LRAs include small organic molecule compounds as well as proteins. In particular, LRAs are those molecules which may be recognized or agonize by various pattern recognition receptors or sensors which may trigger an immune signal response, and which are generally a component of the innate immune system. These include receptors and/or sensors which can recognize extra cellular molecules or DNA/RNA as well as cytosolic DNA/RNA.

These LRAs may be classed according the receptor or sensor system by which they are recognized, with some agents, or ligands, being recognized by a multiple receptor or sensor systems. In some embodiments, LRAs for use herein may be one or more pathogen recognition receptor agonists (e.g., prostratin, bryostratin, ingenol, TNF-alpha and cytokine/cytokine-based complexes such as IL-15, IL-15 superagonist). In some aspects, pathogen recognition receptor agonists herein can activate toll-like receptors, RIG-1-like receptors, cytosolic DNA sensors, are cyclic dinucleotides, or any combination thereof. Non-limiting RIG-I-like receptor ligands suitable for use herein include 5′ppp-dsRNA, a specific agonist of RIG-I; 3p-hpRNA, a specific agonist of RIG-I; Poly(I:C)/LyoVec complexes that are recognized by RIG-I; Poly(dA:dT)/LyoVec complexes that are indirectly recognized by RIG-I; MDA-5; and the like. In some examples, the choice of RIG-I-like receptor ligand(s) for use herein may depend on the presence of poly I:C in the composition or the size of poly I:C in the composition.

In some aspects, cyclic dinucleotides (CDNs; also referred to as STING ligands (Stimulator of IFN genes)) for use herein can be natural CDNs (e.g., 2′3′-cGAMP, 3′3′-cGAMP, c-di-AMP, c-di-GMP); cAIM-derived CDNs (e.g., cAIMP (CL592), cAIMP Difluor (CL614), cAIM(PS)2 Difluor (Rp/Sp) (CL656)); cGAMP-derived CDNs (e.g., 2′2′-cGAMP, 2′3′-cGAM(PS)2 (Rp/Sp), 3′3′-cGAMP Fluorinated, c-di-AMP-derived CDNs, c-di-AMP Fluorinated, 2′3′-c-di-AMP, 2′3′-c-di-AM(PS)2 (Rp,Rp)); c-di-GMP-derived CDNs (e.g., c-di-GMP Fluorinated, 2′3′-c-di-GMP); c-di-IMP-derived CDNs (e.g., c-di-IMP); or any combination thereof. In some aspects, CDS (cytosolic DNA sensors) for use herein may be, but are not limited to, dsDNA-EC, G3-YSD, HSV-60, ISD, ODN TTAGGG (A151), Poly(dA:dT), Poly(dG:dC), VACV-70, and the like.

In some embodiments, LRAs for use herein may include any of those which may be recognized or modulate the toll-like receptors (TLRs). TLRs are transmembrane proteins which can detect extra cellular LRAs. There are a number of known TLRs each one devoted to recognizing a distinct molecular pattern, for example ten in humans and three known in mice. Suitable TLR agonists include, but are not limited to agonists for TLR-2, TLR-3, TLR-4, TLR-5, TLR-7, TLR-8, TLR-9. In some examples, TLR agonists may include the TLR-3 agonists such as double stranded RNA and/or Poly I:C, of high and low molecular weights, TLR-5 agonists such as flagellin (bacterial), TLR-7/8 agonists such as ssRNA, TLR-7 agonists such as GS-9620, TLR-7/8 agonist R-848, and TLR-9 agonists such as CpG-ODNs. The activation of the TRLs may cause an immunologic response which may induce latency reversal of the HIV latent virus.

In some embodiments, the LRAs for use in the compositions herein is polyinosinic:polycytidylic acid (Poly I:C).

(d) Biofunctionalized Nanocomposites

In various embodiments, compositions disclosed herein comprise a biofunctionalized nanocomposite. As used herein, the term “nanocomposite” refers to a composition comprised of a nanoparticle core partially or completely surrounded with a material. As used herein, the term “biofunctionalized nanocomposite” refers to a nanocomposite that has been modified to add at least one biological function.

In various embodiments, biofunctionalized nanocomposites of the present disclosure can have a core comprising a nanoparticle formed of Prussian blue materials and a shell obtained by partially or completely encapsulating the Prussian blue core with a biocompatible coating wherein at least one biomolecule is attached to, or absorbed to, the biocompatible coating.

In various embodiments, biofunctionalized nanocomposites disclosed herein may comprise a core comprising a nanoparticle formed of Prussian blue materials and a shell obtained by partially or completely encapsulating the Prussian blue core with a biocompatible coating. In some aspects, a shell encapsulates about 25%, about 50%, about 75%, or about 100% of the nanoparticle. In a preferred aspect, the shell completely encapsulates a nanoparticle formed of Prussian blue materials as disclosed herein.

In various embodiments, biofunctionalized nanocomposites disclosed herein may comprise a biocompatible coating prepared as described herein. In some aspects, a shell may be formed of one or more of the biocompatible coating materials disclosed herein. In other aspects, a biocompatible coating suitable for use in a biofunctionalized nanocomposite disclosed herein may be citrate, dextran, chitosan, silica, polyethylene glycol (PEG), avidin, a protein, a nucleic acid, a carbohydrate, a lipid, neutravidin, streptavidin, gelatin, collagen, fibronectin, albumin, a serum protein, a lysozyme, a phospholipid, a polyvinyl pyrrolidone (PVP), a polyvinyl alcohol, polyethylene glycol diacrylate, polyethylenimine (PEI), or a combination thereof. In other aspects, a biocompatible coating may comprise PEI having an average molecular weight (MW) in the range from about 100 daltons to about 100,000 daltons. In some aspects, PEI may have an average molecular weight of about 100 daltons, about 500 daltons, about 1,000 daltons, about 1,500 daltons, about 2,000 daltons, about 2,500 daltons, about 3,000 daltons, about 3,500 daltons, about 4,000 daltons, about 4,500 daltons, about 5,000 daltons, about 6,000 daltons, about 7,000 daltons, about 8,000 daltons, about 9,000 daltons, about 10,000 daltons, about 20,000 daltons, about 30,000 daltons, about 40,000 daltons, about 50,000 daltons, about 60,000 daltons, about 70,000 daltons, about 80,000 daltons, about 90 000 daltons, or about 100,000 daltons. In yet other aspects, a biocompatible coating may comprise PEI polymers of at least two different average molecular weights ranging from about 100 daltons to about 100,000 daltons. In other aspects, a biocompatible coating may comprise PEI polymers of at least two different average molecular weights wherein there molecular weight may be about 100 daltons, about 500 daltons, about 1,000 daltons, about 1,500 daltons, about 2,000 daltons, about 2,500 daltons, about 3,000 daltons, about 3,500 daltons, about 4,000 daltons, about 4,500 daltons, about 5,000 daltons, about 6,000 daltons, about 7,000 daltons, about 8,000 daltons, about 9,000 daltons, about 10,000 daltons, about 20,000 daltons, about 30,000 daltons, about 40,000 daltons, about 50,000 daltons, about 60,000 daltons, about 70,000 daltons, about 80,000 daltons, about 90 000 daltons, about 100,000 daltons, or a combination thereof.

In various embodiments, biofunctionalized nanocomposites disclosed herein may comprise a shell partially or completely encapsulating a nanoparticle with biocompatible coating wherein at least one biomolecule may be attached to, or absorbed to, the biocompatible coating. In various embodiments, the biocompatible coating disclosed herein may absorb at least 25%, at least 50%, or at least 75% biomolecule weight by total weight of the biocompatible coating. In other embodiments, at least 25%, at least 50%, at least 75%, or at least 100% of the outer surface of the biocompatible coating comprises attached biomolecules.

In some aspects, biomolecules may be one or more of the biomolecules disclosed herein. In an aspect, a biomolecule suitable for use in a biofunctionalized nanocomposite disclosed herein may be an antibody, a peptide, a protein, an enzyme, an amino acid, a nucleic acid, a carbohydrate, a fat, an aptamer, a small molecule, a synthetic molecule or a combination thereof. In preferred aspects, a biomolecule suitable for use in a biofunctionalized nanocomposite disclosed herein may be a LRA, a bnAB, or a combination thereof.

In various embodiments, a biofunctionalized nanocomposite disclosed herein can be stable. As used herein, a biofunctionalized nanocomposite is considered to be “stable” when the neither the heating ability nor the hydrodynamic diameter of the composition have changed from baseline measurements.

In some embodiments, a biofunctionalized nanocomposite disclosed herein can be stable from about 20° C. to about 120° C. In some aspects, a biofunctionalized nanocomposite can be stable at no less than about 20° C., about 40° C., about 60° C., about 80° C., about 100° C., or about 120° C. In other aspects, a biofunctionalized nanocomposite can be stable no less than about 80° C.

In other embodiments, a biofunctionalized nanocomposite disclosed herein can be stable from about 3 days to about 14 days. In an aspect, a biofunctionalized nanocomposite can be stable for about 3 days, about 5 days, about 7 days, about 10 days, about 12 days, or about 14 days. In another aspect, a biofunctionalized nanocomposite can be stable for about 7 days. In yet other embodiments, a biofunctionalized nanocomposite as disclosed herein can be stable from about 0° C. to about 90° C. In an aspect, a biofunctionalized nanocomposite can be stable at about 0° C., about 10° C., about 20° C., about 30° C., about 40° C., about 50° C., about 60° C., about 70° C., about 80° C., or about 90° C. In another aspect, a biofunctionalized nanocomposite can be stable at about 4° C., about 20° C., or about 80° C. In still other embodiments, a biofunctionalized nanocomposite as disclosed herein can be stable from about 0° C. to about 90° C. for up to about 14 days. In an aspect, a biofunctionalized nanocomposite can be stable from about 0° C. to about 90° C. for about 3 days, about 5 days, about 7 days, about 10 days, about 12 days, or about 14 days. In another aspect, a biofunctionalized nanocomposite can be stable at about 0° C., about 10° C., about 20° C., about 30° C., about 40° C., about 50° C., about 60° C., about 70° C., about 80° C., or about 90° C. for about 3 days, about 5 days, about 7 days, about 10 days, about 12 days, or about 14 days. In preferred aspects, a biofunctionalized nanocomposite can be stable at about 4° C., about 20° C., or about 80° C. for about 7 days.

### II. Pharmaceutical Compositions

In some embodiments, nanoparticles disclosed herein for use according to the methods herein described may be provided per se or as part of a pharmaceutical composition, where the nanoparticles can be mixed with suitable carriers or excipients. In some embodiments, compositions disclosed herein can include one or more nanoparticles disclosed herein. In certain embodiments, nanoparticles herein can be combined with a pharmaceutically acceptable carrier (excipient) to form a pharmaceutical composition for use in treating HIV in a subject.

In some embodiments, pharmaceutical compositions disclosed herein can include at least one nanoparticle having at least one biocompatible coating. In some embodiments, pharmaceutical compositions disclosed herein can include at least one nanoparticle having at least one biocompatible coating such as a citrate coating, a PEI coating, or both. In some embodiments, pharmaceutical compositions disclosed herein can include at least one nanoparticle having at least two biocompatible coatings. In some embodiments, pharmaceutical compositions disclosed herein can include at least one nanoparticle having at least two biocompatible coatings wherein the first coating has and opposite charge from the second coating. In some embodiments, pharmaceutical compositions disclosed herein can include at least one nanoparticle having at least one biomolecule attached to, or absorbed to, at least one of the biocompatible coatings encasing the nanoparticle. In some embodiments, pharmaceutical compositions disclosed herein can include at least one nanoparticle comprising at least one LRA attached, at least one bnAb attached, or any combination thereof.

As used herein a “pharmaceutical composition” refers to a preparation of one or more of the active ingredients described herein (e.g., nanoparticles) with other chemical components such as physiologically suitable carriers and excipients. A purpose of a pharmaceutical composition is to facilitate administration of a compound to an organism. Herein the term “active ingredient” refers to one or more of the nanoparticles (e.g., a nanoparticle having LRA(s) and/or bnAb(s) attached to its coating) disclosed herein.

(i) Pharmaceutically Acceptable Carriers and Excipients

Hereinafter, the phrases “physiologically acceptable carrier” and “pharmaceutically acceptable carrier”, which may be interchangeably used, refer to a carrier or a diluent that does not cause significant irritation to an organism and does not abrogate the biological activity and properties of the administered compound. An adjuvant is included under these phrases.

In various embodiments, compositions disclosed herein may further compromise one or more pharmaceutically acceptable diluent(s), excipient(s), or carrier(s). As used herein, a pharmaceutically acceptable diluent, excipient, or carrier, refers to a material suitable for administration to a subject without causing undesirable biological effects or interacting in a deleterious manner with any of the components of the composition in which it is contained. Pharmaceutically acceptable diluents, carriers, and excipients can include, but are not limited to, physiological saline, Ringer's solution, phosphate solution or buffer, buffered saline, and other carriers known in the art. Pharmaceutical compositions may also include stabilizers, anti-oxidants, colorants, other medicinal or pharmaceutical agents, carriers, adjuvants, preserving agents, stabilizing agents, wetting agents, emulsifying agents, solution promoters, salts, solubilizers, antifoaming agents, antioxidants, dispersing agents, surfactants, and combinations thereof. Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. Techniques for formulation and administration of drugs may be found in REMINGTON'S PHARMACEUTICAL SCIENCES, Mack Publishing Co., Easton, Pa., latest edition, which is incorporated herein by reference.

In various embodiments, pharmaceutical compositions described herein may be formulated in conventional manner using one or more physiologically acceptable carriers comprising excipients and auxiliaries to facilitate processing of genetically modified endothelial progenitor cells into preparations which can be used pharmaceutically. In other embodiments, any of the well-known techniques, carriers, and excipients may be used as suitable and as understood in the art.

In various embodiments, pharmaceutical compositions described herein may be an aqueous suspension comprising one or more polymers as suspending agents. In some aspects, polymers that may comprise pharmaceutical compositions described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In other aspects, compositions disclosed herein may comprise at least 5%, at least 10%, at least 20%, at least 25%, at least 30%, at least 35%, at least 40%, at least 45%, at least 50% total amount of polymers as suspending agent(s) by total weight of the composition.

In various embodiments, pharmaceutical compositions disclosed herein may comprise a viscous formulation. In some aspects, viscosity of the composition may be increased by the addition of one or more gelling or thickening agents. In other aspects, compositions disclosed herein may comprise one or more gelling or thickening agents in an amount to provide a sufficiently viscous formulation to remain on treated tissue. In still other aspects, compositions disclosed herein may comprise at least 5%, at least 10%, at least 20%, at least 25%, at least 30%, at least 35%, at least 40%, at least 45%, at least 50% total amount of gelling or thickening agent(s) by total weight of the composition. In yet other aspects, suitable thickening agents can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or combinations thereof. In some embodiments, suitable thickening agent may be carboxymethylcellulose.

In various embodiments, pharmaceutical compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some aspects, pharmaceutical compositions disclosed herein may comprise at least 5%, at least 10%, at least 20%, at least 25%, at least 30%, at least 35%, at least 40%, at least 45%, at least 50% total amount of one or more agents by total weight of the composition. In other aspects, one or more of these agents may be added to improve the performance, efficacy, safety, shelf-life and/or other property of the muscarinic antagonist composition of the present disclosure. In s aspects, additives will be biocompatible, and will not be harsh, abrasive, or allergenic.

In various embodiments, pharmaceutical compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some aspects, any pharmaceutically acceptable organic or inorganic acid may be used. In other aspects, compositions disclosed herein may comprise at least 5%, at least 10%, at least 20%, at least 25%, at least 30%, at least 35%, at least 40%, at least 45%, at least 50% total amount of one or more acidifying agents by total weight of the composition.

In various embodiments, pharmaceutical compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some aspects, any pharmaceutically acceptable organic or inorganic base can be used. In other aspects, compositions disclosed herein may comprise at least 5%, at least 10%, at least 20%, at least 25%, at least 30%, at least 35%, at least 40%, at least 45%, at least 50% total amount of one or more alkalizing agents by total weight of the composition.

In various embodiments, pharmaceutical compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and other materials known to one of ordinary skill in the art. In some aspects, compositions disclosed herein may comprise at least 5%, at least 10%, at least 20%, at least 25%, at least 30%, at least 35%, at least 40%, at least 45%, at least 50% total amount of one or more antioxidants by total weight of the composition.

In other embodiments, pharmaceutical compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some aspects, any pharmaceutically acceptable organic or inorganic buffer can be used. In another aspect, compositions disclosed herein may comprise at least 5%, at least 10%, at least 20%, at least 25%, at least 30%, at least 35%, at least 40%, at least 45%, at least 50% total amount of one or more buffering agents by total weight of the composition. In other aspects, the amount of one or more buffering agents may depend on the desired pH level of a composition. In some embodiments, pharmaceutical compositions disclosed herein may have a pH of about 6 to about 9. In other embodiments, pharmaceutical compositions disclosed herein may have a pH greater than about 8, greater than about 7.5, greater than about 7, greater than about 6.5, or greater than about 6. In a preferred embodiment, compositions disclosed herein may have a pH greater than about 6.8.

In various embodiments, pharmaceutical compositions disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin, Nipasol, isopropyl alcohol and a combination thereof. In some aspects, any pharmaceutically acceptable preservative can be used. In other aspects, pharmaceutical compositions disclosed herein may comprise at least 5%, at least 10%, at least 20%, at least 25%, at least 30%, at least 35%, at least 40%, at least 45%, at least 50% total amount of one or more preservatives by total weight of the composition.

In other embodiments, pharmaceutical compositions disclosed herein may comprise one or more surface-acting reagents or detergents. In some aspects, surface-acting reagents or detergents may be synthetic, natural, or semi-synthetic. In other aspects, compositions disclosed herein may comprise anionic detergents, cationic detergents, zwitterionic detergents, ampholytic detergents, amphoteric detergents, nonionic detergents having a steroid skeleton, or a combination thereof. In still other aspects, pharmaceutical compositions disclosed herein may comprise at least 5%, at least 10%, at least 20%, at least 25%, at least 30%, at least 35%, at least 40%, at least 45%, at least 50% total amount of one or more surface-acting reagents or detergents by total weight of the composition.

In various embodiments, pharmaceutical compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some aspects, pharmaceutical compositions disclosed herein may comprise at least 5%, at least 10%, at least 20%, at least 25%, at least 30%, at least 35%, at least 40%, at least 45%, at least 50% total amount of one or more stabilizers by total weight of the composition.

In other embodiments, pharmaceutical compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In preferred embodiments, a vapor pressure depression method is used to calculate the osmolarity of the compositions disclosed herein. In some aspects, the amount of one or more tonicity agents comprising a pharmaceutical composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In other aspects, a composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some embodiments, a pharmaceutical composition described herein has an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In still other aspects, pharmaceutical compositions disclosed herein may comprise at least 5%, at least 10%, at least 20%, at least 25%, at least 30%, at least 35%, at least 40%, at least 45%, at least 50% total amount of one or more tonicity modifiers by total weight of the composition.

(ii) Dosage Formulations

Suitable routes of administration may, for example, include oral, rectal, transmucosal, especially transnasal, intestinal or parenteral delivery, including intramuscular, subcutaneous and intramedullary injections as well as, intravenous, intraperitoneal, intranasal injections.

One may administer the pharmaceutical compositions herein in a local or systemic manner, for example, via local injection of the pharmaceutical composition directly into a tissue region of a patient. In some embodiments, a pharmaceutical composition disclosed herein can be administered parenterally, e.g., by intravenous injection, intracerebroventricular injection, intra-cisterna magna injection, intra-parenchymal injection, or a combination thereof. In some embodiments, a pharmaceutical composition disclosed herein can administered to the human patient via at least two administration routes. In some examples, the combination of administration routes by be intramuscular injection and intravenous injection; subcutaneous injection and intravenous injection; intracerebroventricular injection and intravenous injection; intrathecal injection and intravenous injection; intra-cisterna magna injection and intravenous injection; and intra-parenchymal injection and intravenous injection. In some embodiments, a pharmaceutical composition disclosed herein can administered to a cancer cell, a tumor, or both. In some embodiments, a pharmaceutical composition disclosed herein can administered to a cancer cell, a tumor, or both by introducing the cancer cell, the tumor, or both to a pharmaceutical composition disclosed herein.

Pharmaceutical compositions of the present disclosure may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present disclosure may be formulated in conventional manner using one or more physiologically acceptable carriers comprising excipients and auxiliaries, which facilitate processing of the active ingredients (e.g., a nanoparticle having LRA(s) and/or bnAb(s) attached to its coating) into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, tone or more active ingredients (e.g., a nanoparticle having LRA(s) and/or bnAb(s) attached to its coating) may be in powder form for constitution with a suitable vehicle, e.g., sterile, pyrogen-free water based solution, before use.

Pharmaceutical compositions suitable for use in context of the present disclosure can include compositions wherein the active ingredients are contained in an amount effective to achieve the intended purpose. In some embodiments, a therapeutically effective amount means an amount of active ingredients (e.g., a nanoparticle having LRA(s) and/or bnAb(s) attached to its coating) effective to prevent, slow, alleviate or ameliorate symptoms of a disorder (e.g., HIV) or prolong the survival of the subject being treated.

Determination of a therapeutically effective amount is well within the capability of those skilled in the art, especially in light of the detailed disclosure provided herein.

For any preparation used in the methods of the present disclosure, the therapeutically effective amount or dose can be estimated initially from in vitro and cell culture assays and or screening platforms disclosed herein. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in THE PHARMACOLOGICAL BASIS OF THERAPEUTICS, Ch. 1 p. 1).

Dosage amount and interval may be adjusted individually to brain or blood levels of the active ingredients and/or one of its metabolites are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc. Effective doses may be extrapolated from dose-responsive curves derived from in vitro or in vivo test systems.

### III. Methods

Embodiments of the instant disclosure relate to novel methods and compositions for treating HIV in a subject. The present disclosure also provides methods of reducing and/or eradicating latent HIV reservoirs in a subject.

The subject to be treated by the methods described herein can be a mammal, more preferably a human. Mammals include, but are not limited to, farm animals, sport animals, pets, primates, horses, dogs, cats, mice and rats. In some examples, the subject may have, be at risk for, or be suspected of having, a target disease/disorder characterized as a retrovirus infections. Retroviruses are a family of enveloped viruses that replicate in a host cell through the process of reverse transcription. A retrovirus is a single-stranded positive sense RNA virus with a DNA intermediate and, as an obligate parasite, targets a host cell. In some non-limiting examples, a retrovirus to be treated by the methods herein may be HTLV 1 (e.g., T-cell leukaemias/lymphomas, Tropical spastic paraparesis), HTLV 2, HIV 1 & 2-AIDS and HTLV-3 and HTLV-4. A subject having a retrovirus infection or suspected of having a retrovirus infection can be identified by routine medical examination, e.g., laboratory tests, organ functional tests, or diagnostic screening. In some examples, a subject has an active HIV infection or is suspected of having such an infection. In some examples, a subject has HIV or is suspected of having HIV. In some examples, a subject has AIDS or is suspected of having AIDS. In some examples, a subject has latent HIV or is suspected of having latent HIV.

As used herein, “an effective amount” refers to the amount of each active agent required to confer therapeutic effect on the subject, either alone or in combination with one or more other active agents. Determination of whether an amount of biofunctionalized nanocomposites (e.g., nanoparticles, PBNPs) disclosed herein achieved the therapeutic effect would be evident to one of skill in the art. Effective amounts vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

In some embodiments, methods of treating a subject with a retrovirus infection may comprise administering any one of the biofunctionalized nanocomposites (e.g., nanoparticles, PBNPs) to the subject, thereby treating the retroviral infection. In some embodiments, methods herein may include administration any one of the biofunctionalized nanocomposites (e.g., nanoparticles, PBNPs) to treat HIV/AIDS.

The human immunodeficiency virus (HIV) causes the acquired immunodeficiency syndrome (AIDS), a condition in humans in which progressive failure of the immune system allows life-threatening opportunistic infections and cancers to thrive. In some embodiments, methods herein may treat HIV by improving one or more symptoms of the disease. In some aspects, methods herein may treat HIV by reversing HIV latency. The term “latent HIV,” as used herein, includes a state of the HIV life cycle in which the HIV genome has integrated into the chromosomal DNA of the infected cell, but replication of the genome and HIV proliferation in the infected cell is dormant. Where methods herein reverse HIV latency, embodiments herein may trigger one or more symptoms of HIV. Symptoms of HIV to be treated and/or triggered herein can be acute HIV symptoms (e.g., fever, headaches, sore throat, excessive fatigue, chills, muscle pain, swollen lymph nodes) and/or chronic HIV symptoms (e.g., coughing, strain in breathing, weight loss, diarrhea, fatigue, high fever).

In some embodiments, methods herein may comprise administration of any one of the biofunctionalized nanocomposites (e.g., nanoparticles, PBNPs) or pharmaceutical compositions containing thereof as disclosed herein to a subject for the treatment of an HIV infection that leads to the reduction or elimination of the virus from latent reservoirs in infected resting cells such as central and/or transitional memory CD4+ T cells. In some embodiments, methods herein may comprise administration of any one of the biofunctionalized nanocomposites (e.g., nanoparticles, PBNPs) or pharmaceutical compositions containing thereof as disclosed herein to a subject for the treatment of a viral infection such as an HIV infection that leads to the reactivation of virally infected cells from latent reservoirs and activation of the subject's immune system to combat and kill the recently reactivated virally infected cells.

In some embodiments, methods herein may treat HIV by improving the CD4 count in a subject having or suspected of having HIV. CD4 cells are a type of white blood cell. The CD4 count of an uninfected adult/adolescent who is generally in good health ranges from about 500 cells/mm³ to about 1,600 cells/mm³. In contrast, if HIV has destroyed so many CD4 cells that the subject has a CD4 count of fewer than 200/mm³, the subject is considered to have progressed to stage 3 (AIDS), the most advanced stage of HIV infection. In some embodiments, methods herein may treat HIV by increasing and/or maintaining the CD4 count in a subject at or above about 200/mm³ to about 1,600 cells/mm³, about 200/mm³ to about 1,000 cells/mm³, or about 200/mm³ to about 500 cells/mm³.

In some embodiments, methods of reversing human immunodeficiency virus (HIV) latency in a cell may comprise administering any one of the biofunctionalized nanocomposites (e.g., nanoparticles, PBNPs) to a subject in need thereof, thereby reversing the HIV latency. In some embodiments, methods of reversing human immunodeficiency virus (HIV) latency in a cell may increase HIV viral load. Methods of measuring the amount (viral load) of HIV genetic material (RNA) in the blood are known in the art and are generally reported as the number of HIV copies in a milliliter (copies/mL) of blood. In some embodiments, methods herein can increase HIV viral load in a subject that is positive for HIV and has viral loads that are consistently less than 200 copies/mL. In some embodiments, methods herein can increase HIV viral load in a subject that is positive for HIV and has viral loads that are consistently less than 200 copies/mL for about 1 year to about 20 years, about 2 years to about 15 years, to about 3 years to about 10 years. In some embodiments, methods herein can increase HIV viral load in a subject that is positive for HIV and has not presented with a symptom of HIV infection for about 1 year to about 20 years, about 2 years to about 15 years, to about 3 years to about 10 years.

Conventional methods, known to those of ordinary skill in the art of medicine, can be used to administer the compositions disclosed herein to a subject, depending upon the type of disease to be treated or the site of the disease. In some embodiments, compositions herein can be administered to a subject by intravenous infusion by subcutaneous administration, by inhalation, by intranasal administration or other mode of administration. In some embodiments, compositions herein can be administered to a subject orally.

In some embodiments, any of the methods disclosed herein can further include monitoring occurrence of one or more adverse effects in the subject. Exemplary adverse effects include, but are not limited to, hepatic impairment, hematologic toxicity, neurologic toxicity, cutaneous toxicity, gastrointestinal toxicity, or a combination thereof. When one or more adverse effects are observed, the method disclosed herein can further include reducing or increasing the dose of one or more of the disclosed biofunctionalized nanocomposites (e.g., nanoparticles, PBNPs) disclosed herein on the adverse effect or effects in the subject. For example, when a moderate to severe hepatic impairment is observed in a subject after treatment, compositions of use to treat the subject can be reduced in concentration or frequency of dosing with one or more disclosed compounds.

In some embodiments, one or more of the biofunctionalized nanocomposites (e.g., nanoparticles, PBNPs) disclosed herein can be administered concurrently with the one or more anti-retroviral therapeutics by the same or different modes of administration. In some aspects, an anti-retroviral therapeutic suitable for concurrent use with any of the compositions herein may be enfuvirtide, zidovudine, abacavir, lamivudine, emtricitabine, tenfovir, nevirpine, efavirenz, etravirine, rilpivirine, raltegravir, elvitegravir, dolutegravir, lopinavir, indinavir, nelfinavir, amprenavir, ritonavir, darunavir, atazanavir, bevirimat, vivecon, stavudine, didanosine, delavirdine, nevirapine, fosamprenavir, saquinavir, tipranavir, maraviroc or any combination thereof. In some aspects, an anti-retroviral therapeutic suitable for concurrent use with the any of the compositions herein may be a nucleoside/nucleotide reverse transcriptase inhibitor (NRTI), non-nucleoside reverse transcriptase inhibitor (NNRTI), protease inhibitor, fusion or entry inhibitor, integrase inhibitor or a combination thereof.

In some embodiments, one or more disclosed compositions (e.g., nanoparticles, PBNPs) herein can be administered before, during or after the one or more anti-retroviral therapeutics. In other embodiments, the one or more anti-retroviral therapeutics can be administered systemically. In certain embodiments, the one or more anti-retroviral therapeutics can be administered locally directly to one or more tumors in the subject. In some embodiments, the one or more anti-retroviral therapeutics can be administered by intravenous administration, e.g., as a bolus or by continuous infusion over a period of time, by intramuscular, intraperitoneal, intracerebrospinal, subcutaneous, intra-arterial, intra-articular, intrasynovial, intrathecal, intratumoral, oral, inhalation or topical routes. In other embodiments, the one or more anti-retroviral therapeutics can be administered to the subject by intravenous infusion.

An effective amount of the pharmaceutical composition described herein can be administered to a subject (e.g., a human) in need of the treatment via a suitable route, systemically or locally. In some embodiments, one or more disclosed compositions (e.g., nanoparticles, PBNPs) can be administered by intravenous administration, e.g., as a bolus or by continuous infusion over a period of time, by intramuscular, intraperitoneal, intracerebrospinal, subcutaneous, intra-arterial, intra-articular, intrasynovial, intrathecal, intratumoral, oral, inhalation or topical routes. In some embodiments, one or more disclosed compositions (e.g., nanoparticles, PBNPs) can be administered intravenously.

In some embodiments, a composition disclosed herein may be administered to a subject in need thereof once. In some embodiments, a composition disclosed herein may be administered to a subject in need thereof more than once. In some embodiments, a first administration of a composition disclosed herein may be followed by a second administration of a composition disclosed herein. In some embodiments, a first administration of a composition disclosed herein may be followed by a second and third administration of a composition disclosed herein. In some embodiments, a first administration of a composition disclosed herein may be followed by a second, third, and fourth administration of a composition disclosed herein. In some embodiments, a first administration of a composition disclosed herein may be followed by a second, third, fourth, and fifth administration of a composition disclosed herein.

In some embodiments, the desired daily dose of compositions disclosed herein may be presented in a single dose or as divided doses administered simultaneously (or over a short period of time) or at appropriate intervals. In various embodiments, administration of a composition disclosed herein may be administered to a subject about once a day, about twice a day, about three times a day. In other embodiments, administration of a composition disclosed herein may be administered to a subject at least once a day, at least once a day for about 2 days, at least once a day for about 3 days, at least once a day for about 4 days, at least once a day for about 5 days, at least once a day for about 6 days, at least once a day for about 1 week, at least once a day for about 2 weeks, at least once a day for about 3 weeks, at least once a day for about 4 weeks, at least once a day for about 8 weeks, at least once a day for about 12 weeks, at least once a day for about 16 weeks, at least once a day for about 24 weeks, at least once a day for about 52 weeks and thereafter.

In some embodiments, administration of a composition disclosed herein may be administered to a subject at least once a week, at least once a week for about 2 weeks, at least once a week for about 3 weeks, at least once a week for about 4 weeks, at least once a week for about 8 weeks, at least once a week for about 12 weeks, at least once a week for about 16 weeks, at least once a week for about 24 weeks, at least once a week for about 52 weeks and thereafter. In a preferred embodiment, administration of a composition disclosed herein may be administered to a subject once a week for at about 12 weeks.

In some embodiments, the amount and/or frequency of compositions disclosed herein administered can be adjusted based upon factors such as the particular compound, disease condition and its severity, according to the particular circumstances surrounding the case, including, e.g., the route of administration, the condition being treated, the target area being treated, and the subject or host being treated. In some aspects, compositions disclosed herein can be administered as many times needed to reactivate a latent virus (e.g., a latent HIV virus) in a subject. In some aspects, compositions disclosed herein can be administered as many times needed to increase the immune response of a subject against a virus (e.g., HIV), a reactivated latent virus (e.g., a latent HIV virus), or a combination thereof.

In some embodiments, biofunctionalized nanocomposites (e.g., nanoparticles, PBNPs) herein may be administered to a subject at an amount suitable for administering between about 0.001 mg/kg and about 50 mg/kg LRA(s), between about 0.01 mg/kg and about 20 mg/kg LRA(s), between about 0.1 and about 10 mg/kg LRA(s), or between about 0.1 mg/kg and about 5 mg/kg LRA(s) body weight per dose to a subject in need thereof. In some embodiments, biofunctionalized nanocomposites (e.g., nanoparticles, PBNPs) herein may be administered to a subject at an amount suitable for administering between about 0.001 mg/kg and about 50 mg/kg bnAb(s), between about 0.01 mg/kg and about 20 mg/kg bnAb(s), between about 0.1 and about 10 mg/kg bnAb(s), or between about 0.1 mg/kg and about 5 mg/kg bnAb(s) body weight per dose to a subject in need thereof.

### IV. Kits

In certain embodiments, the present disclosure provides kits for use in treating a retrovirus (e.g., HIV) in a subject as described herein. In certain embodiments, the present disclosure also provides kits for use in the reduction and/or eradication of latent HIV reservoirs in a subject as described herein. Such kits can include one or more containers including one or more one or more disclosed biofunctionalized nanocomposites, e.g., any of those described. In some embodiments, kits can include one or more containers including one or more one or more disclosed biofunctionalized nanocomposites (e.g., PBNPs), e.g., any one or more anti-retroviral therapeutics disclosed herein.

In some embodiments, the kits herein can include instructions for use in accordance with any of the methods described herein. The included instructions can have a description of administration of the one or more disclosed biofunctionalized nanocomposites (e.g., PBNPs), the one or more anti-retroviral therapeutics, to treat, delay the onset, or alleviate a target disease as those described herein, or a combination thereof. In some embodiments, the kit can further include a description of selecting an individual suitable for treatment based on identifying whether that individual has the target disease, e.g., applying the diagnostic method as described herein. In still other embodiments, the instructions can have a description of administering any one of the compositions described herein to an individual at risk of the target disease.

In some embodiments, kit instructions relating to the use of one or more one or more disclosed biofunctionalized nanocomposites (e.g., PBNPs), one or anti-retroviral therapeutics described herein (e.g., a cisplatin), or a combination thereof can generally include information as to dosage, dosing schedule, and route of administration for the intended treatment. The containers can be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating HIV. In some embodiments, instructions are provided for practicing any of the methods described herein. In some embodiments, instructions are provided for reactivating a latent HIV virus in a subject.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. In some embodiments, a kit has a sterile access port (for example the container can be an intravenous solution bag or a vial having a stopper pierceable by a hypodermic injection needle). In some embodiments, the container also has a sterile access port (for example the container is an intravenous solution bag or a vial having a stopper pierceable by a hypodermic injection needle). At least one active agent in the composition is at least one or more disclosed compounds (e.g., And-1 inhibitors).

In some embodiments, kits herein can optionally provide additional components such as buffers and interpretive information. Normally, the kit comprises a container and a label or package insert(s) on or associated with the container. In some embodiments, the invention provides articles of manufacture comprising contents of the kits described above.

### Examples

The following examples are included to demonstrate preferred embodiments of the disclosure. It should be appreciated by those of skill in the art that the techniques disclosed in the examples that follow represent techniques discovered by the inventors to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

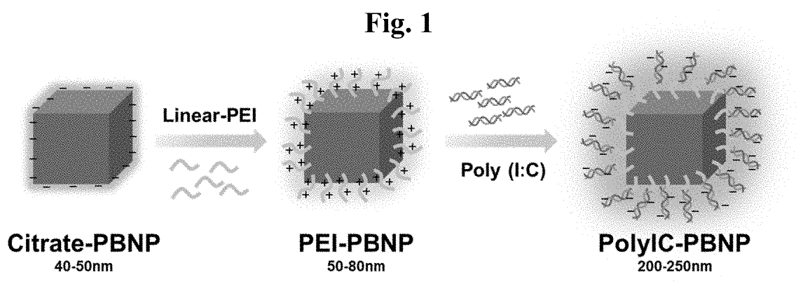

Example 1. To synthesize Prussian blue nanoparticles (PBNPs) as shown in FIG. 1, citrate conjugated PBNP (Citrate-PBNP) were first prepared by co-precipitation method. In brief, an aqueous solution of 1.0 mM FeCl₃·6H₂O and 0.5 mmol citric acid in 20 mL of ultrapure water was added, under vigorous stirring, to an aqueous 20 mL solution containing 1 mM K₄Fe(CN)₆ 3H₂O and 0.5 mmol citric acid at 60° C. After stirring for 1 minute, the solution was allowed to come to room temperature (23° C.±3° C.). The precipitate was isolated from the solution first by the addition of equal volumes of acetone followed by centrifugation at 10,000 revolutions per minute (rpm) for 10 minutes, and finally rinsed by sonication (5 seconds, high power) in ultrapure water. The isolation and rinsing steps were repeated three times before the particles were resuspended by sonication in ultrapure water. Note that all synthetic procedures were conducted using ultrapure water, or “grade 1” water as defined by the International Organization for Standardization (ISO), with resistivity of 18.2 MΩ·cm.

Next, Citrate-PBNPs were surface coated with polyethylenimine (PEI) using a layer-by-layer strategy. In brief, 2 mg of the Citrate-PBNPs were first suspended in 2 mL of 50 wt % polyethylenimine (PEI). Prior to this step, PEI (average MW=2000) was diluted with acetate buffer (pH 5.2) to a concentration of 50 wt %. Next, the PBNP suspension was shaken at room temperature (23° C.±3° C.) for 1 hour, and the PEI-coated PBNPs (PEI-PBNPs) were subsequently collected by centrifugation at 10,000 rpm for 10 minutes with equal volumes of ethanol. After 4 washes with deionized water, the PEI-PBNPs were resuspended by sonication in Milli-Q water. The charged-based PEI coating resulting in the PEI-PBNPs having a positive charge. The PEI-PBNPs were surface coated with polyinosinic-polycytidylic acid (poly I:C) using a layer-by-layer strategy according to the same method as described for PEI coating. The method produced PolyIC-PBNP which had a negative charge.

Example 2. To guarantee that coating PBNPs with poly I:C does not affect the intrinsic characteristics of PBNPs, properties of PolyIC-PBNPs were measured and compared to PEI-PBNPs and Citrate-PBNPs. The size (hydrodynamic diameter) and charge (zeta potential) distributions of Citrate-PBNPs, PEI-PBNPs and PolyIC-PBNPs were measured using dynamic light scattering (DLS) on a Zetasizer Nano ZS (Malvern Instruments). The UV visible absorbance of the nanoparticles were measured on a spectrophotometer.

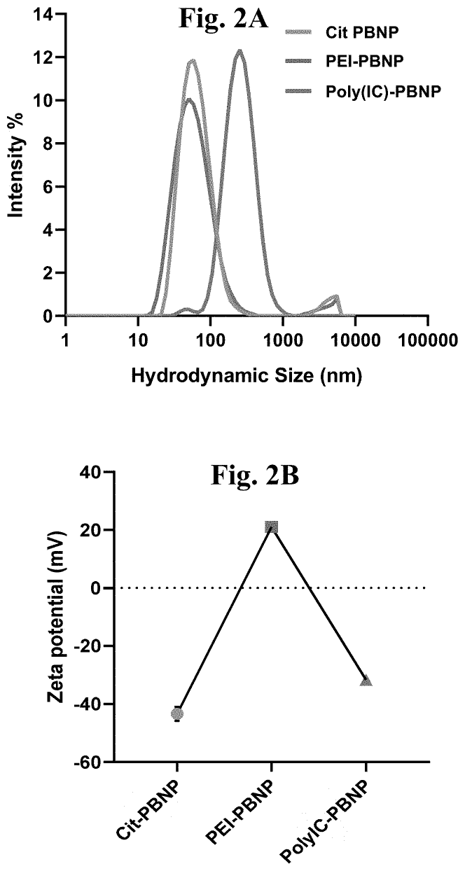

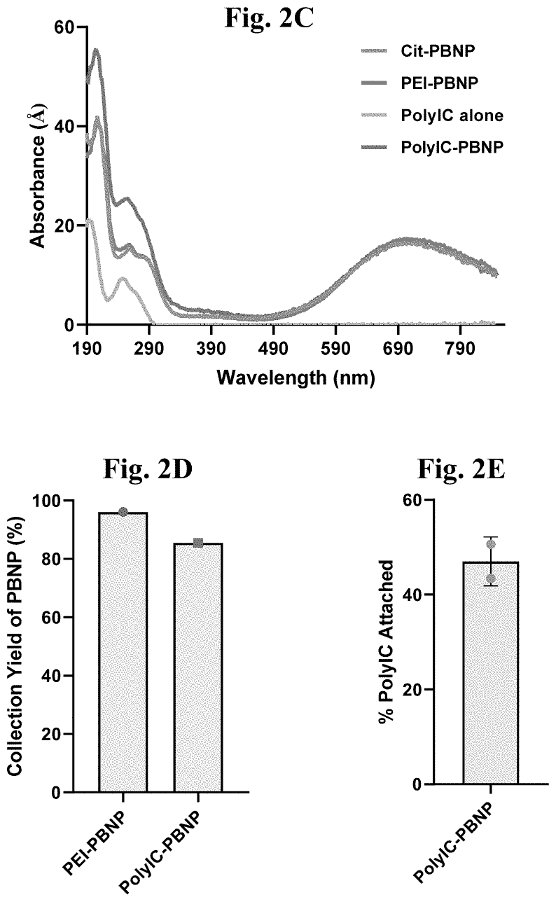

A step-wise increase in nanoparticle size was observed following each coating as characterized by dynamic light scattering (FIG. 2A). The starting Citrate-PBNPs was about 50 nm, the coating of PEI onto Citrate-PBNPs (PEI-PBNP) increased the size to 70-90 nm, after which the coating of poly I:C onto PEI-PBNPs further increased the size to 200-250 nm.

Confirmation that each layer was coated onto the PBNPs was determined by measuring the charge distributions (zeta potential) of these particles (FIG. 2B). Citrate-PBNPs had a negative charge with zeta potential (ZP) of −42 mV. The addition of positively charged PEI changed the charge to a positive charge with a ZP of +25 mV. THe charge of the PEI-PBNPs was changed from a positive charge to a negative charge with a ZP of −36 mV after the PEI-PBNps were coated with poly I:C.

As shown in FIG. 2C, UV-visible spectroscopy revealed a characteristic absorption peak of Citrate-PBNPs ranging from 500 to 900 nm. Coating Citrate-PBNPs with PEI did not change the absorption of the particles (PEI-PBNPs˜500 to 900 nm). The polyIC-PBNPs shows an absorption peak at 500-900 nm and Poly I:C alone had an absorption peak at 260 nm proving effective conjugation of PolyIC onto PBNP.

FIG. 2D shows that the collection yield calculated after coating each layer was 98% and 87% for PBNP. These results showed that there was minimal loss of PBNP after PEI and Poly I:C coating, respectively. Finally, FIG. 2E shows that the poly I:C was attached on PBNP with a coating efficiency of 50%.

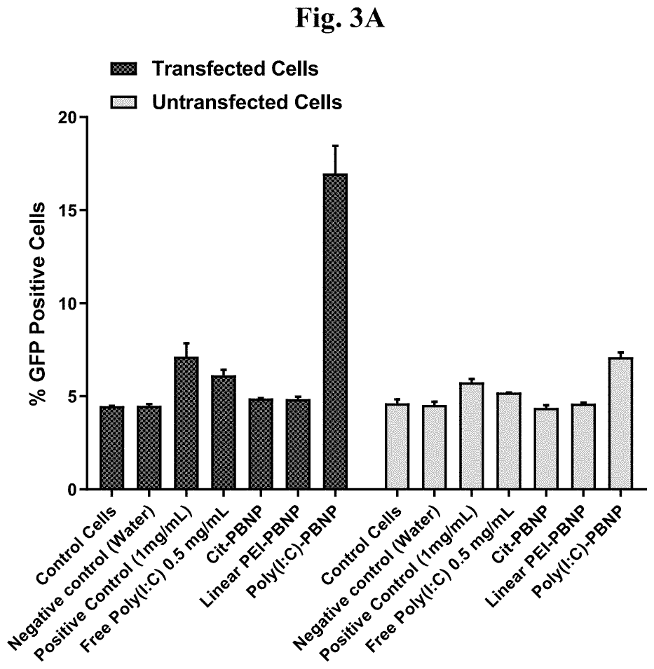

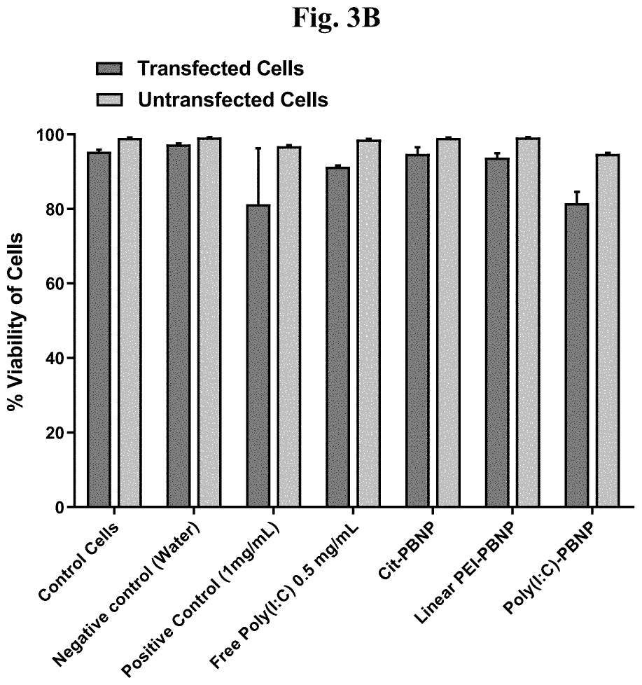

Example 3. To determine if Citrate-PBNPs, PEI-PBNPs, and/or polyIC-PBNPs can reactivate latent HIV virus, the particles were added to model latent HIV cells, J-LAT cells. J-LAT cells harbor latent, transcriptionally competent HIV provirus that encodes green fluorescent protein (GFP) as an indicator of HIV-1 reactivation. FIG. 3A shows that J-LAT cells had improved latency reversal given by the measure of increased GFP production after the cells were incubated with 1 μL ([PBNP]=2 mg/mL, [poly(IC)] attached=0.5 mg/mL) of PolyIC-PBNP compared to equivalent amount of free poly-IC without and with transfection by electroporation. Generally for effective latency reversal, free Poly I:C has to be delivered into the cytoplasm of the latently infected cells via transfection. FIG. 3A shows that poly I:C attached onto the PBNP had better latency reversal even without the aid of transfection. FIG. 3B shows a viability graph indicating no cytotoxicity associated with Citrate-PBNP, PEI-PBNP or polyIC-PBNP on the J-LAT cells.

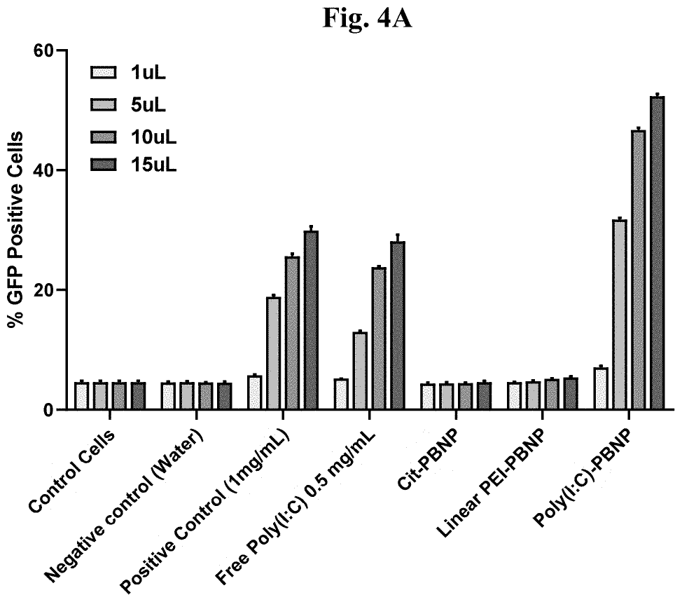

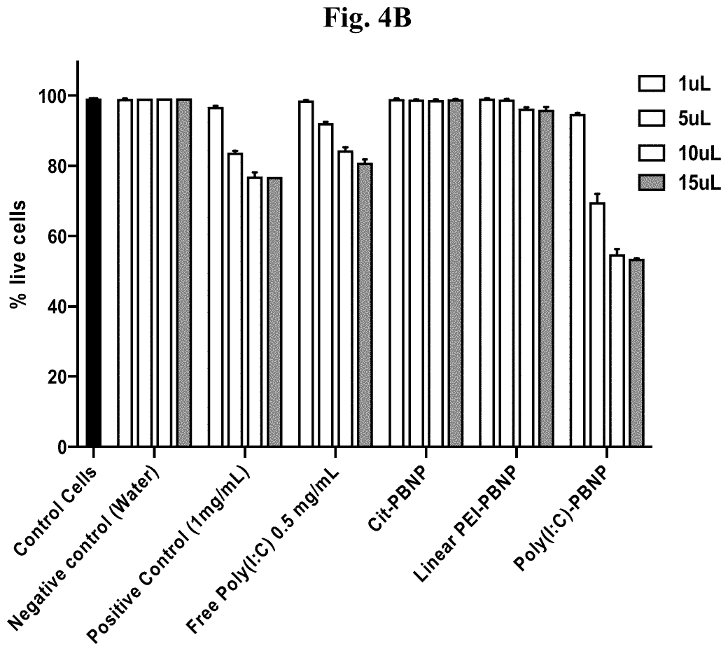

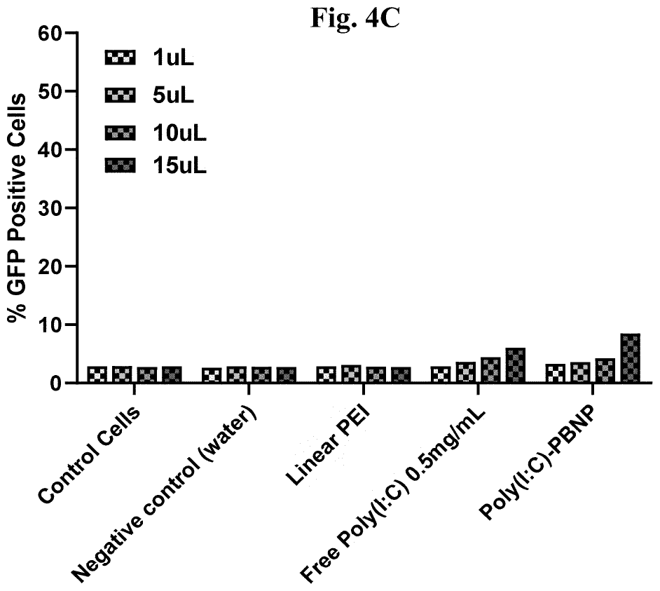

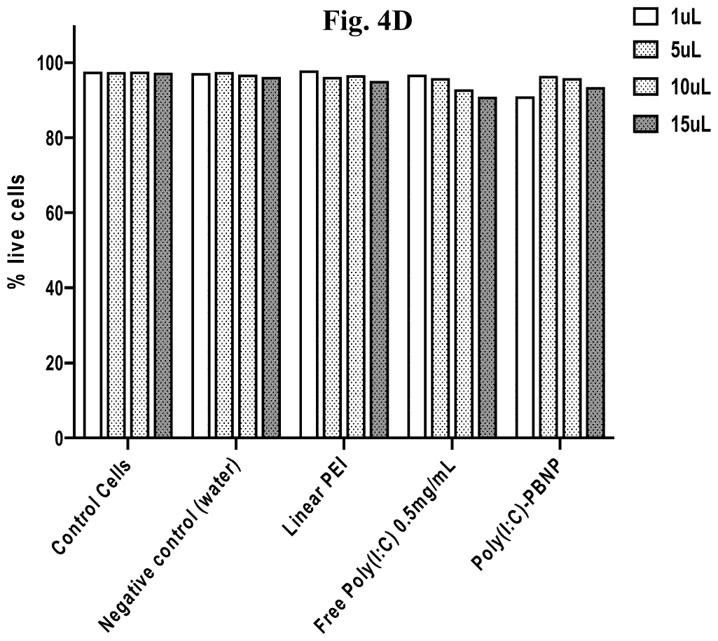

Next, J-LAT cells were treated with increasing concentrations of Citrate-PBNP, PEI-PBNP or polyIC-PBNP (1 μL, 5 μL, 10 μL and 15 μL with [PBNP]=2 mg/mL and [polyIC]=0.5 mg/mL) where the PBNPs were added to the cell media (i.e., the PBNPs were not transfected into the cells). FIG. 4A shows that poly I:C alone reversed latency in a dose dependent manner even without the aid of transfection, but the efficiency of latency reversal, exhibited using JLATs was improved by polyIC-PBNPs compared to its free counterpart. FIG. 4B shows that there was a mild dose dependent cytotoxicity of treated J-LATs, associated with all materials containing poly I:C, showing that poly I:C caused cytotoxicity at higher concentrations.

A major role in antiviral defense is played by mitochondrial antiviral signaling (MAVS) protein, an adaptor protein that that causes oligomerization of the retinoic acid-inducible gene I (RIG-I) and/melanoma differentiation associated gene 5 (MDA5) and the formation of a signalosome sensor to NFkB activation. FIG. 4C shows an F2 cell line lacking MAVS treated with increasing concentrations of polyIC-PBNP (1 μL, 5 μL, 10 μL and 15 μL with [PBNP]=2 mg/mL and [polyIC]=0.5 mg/mL) where the PBNP were added to the cell media (i.e., the PBNPs were not transfected into the cells). F2 cells line lacking MAVS showed very minimal to no GFP expression and latency reversal. This demonstrated Poly I:C reversed latency utilizing MAVS and that the reactivation of HIV was mediated by the cytosolic sensors instead of the toll-like receptor 3 (TLR3) pathway. FIG. 4D shows that the F2 cells lacking MAVS demonstrated no cytotoxicity for all materials even at higher dosage tested (15 μL).

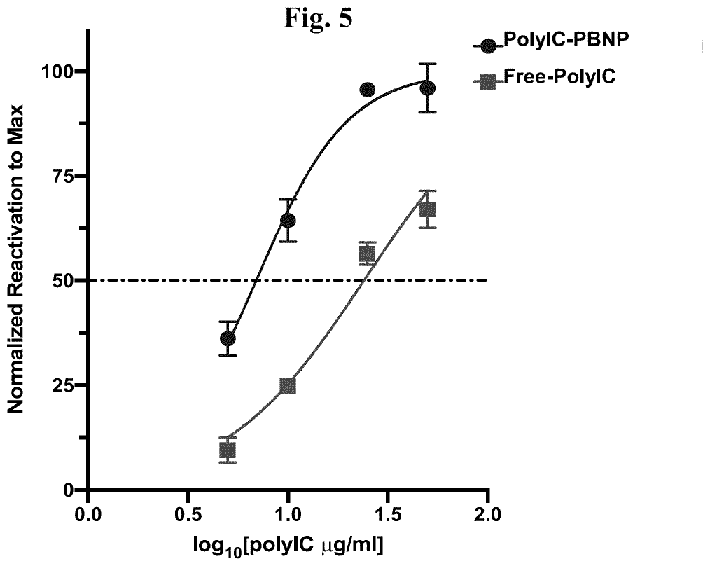

To determine the efficiency of free Poly I:C compared to PolyIC-PBNPs on HIV reactivation, increasing concentrations were added to J-LAT cells. Specifically, nanoparticles (PolyIC-PBNP) were generated at varying Poly I:C concentrations (1.75, 1, 1.5 and 0.75 mg/mL) and added to the cell culture medium to treat the J-LAT cells. The dose-dependence curves on HIV reactivation for free Poly I:C compared to PolyIC-PBNPs is shown in FIG. 5. From these curves, half minimal effective concentration (EC50) was calculated, as provided in Table 1.

TABLE 1

| | |
|---|---|
| Free Poly I:C | PolyIC-PBNPs |

Log (agonist) vs. normalized response - variable slope

| | | |
|---|---|---|
| Best-fit values | | |
| Log EC50 | 0.8416 | 1.380 |
| Hill Slope | 1.904 | 1.239 |
| EC50 | 6.944 | 24.01 |

95% CI (profile likelihood)

| | | |
|---|---|---|
| Log EC50 | 0.7972 to 0.8832 | 1.323 to 1.441 |

The EC50 of PolyIC-PBNPs was 24.01 μg/ml, 3.45 times higher reactivation potency compared to same concentrations of Free-Poly I:C (6.944 μg/ml), after overnight incubation with J-LAT cells. These data showed that Poly I:C when conjugated with PBNP was more effective in eliciting latency reversal effect in HIV-1 latency model, even at lower concentrations compared to its free counterpart, Poly I:C.

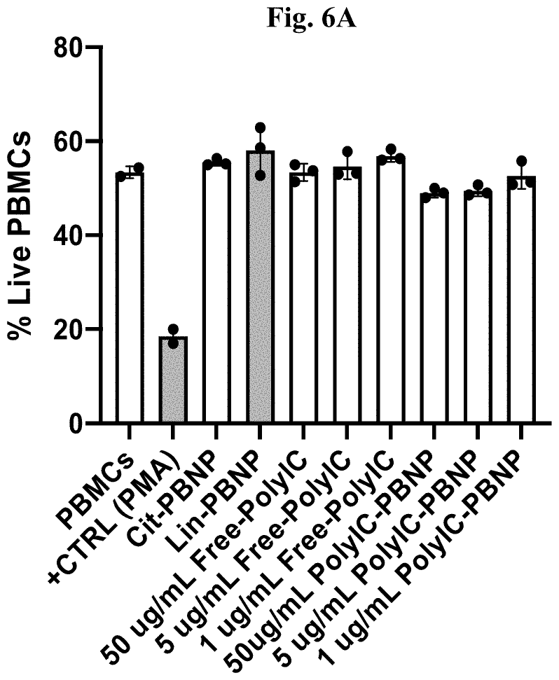

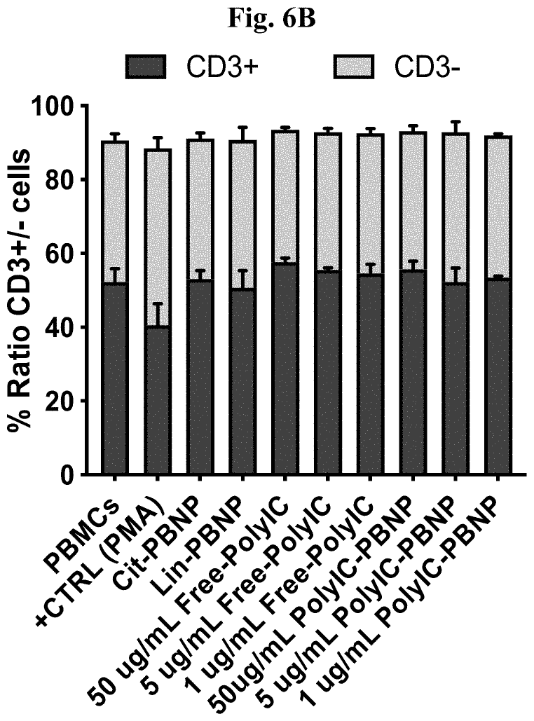

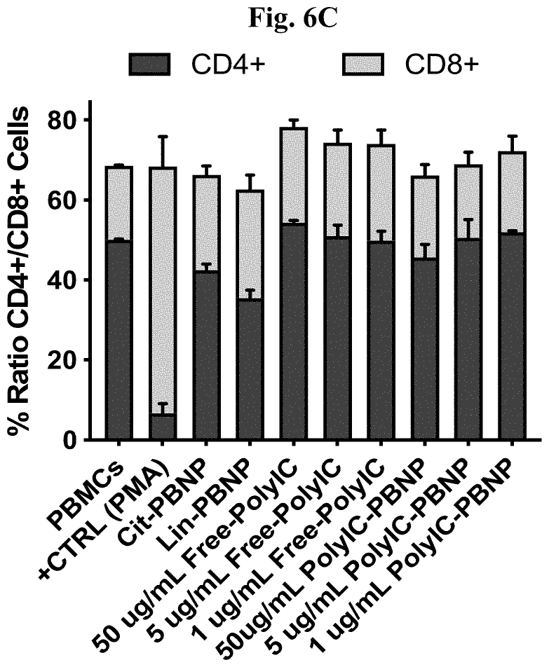

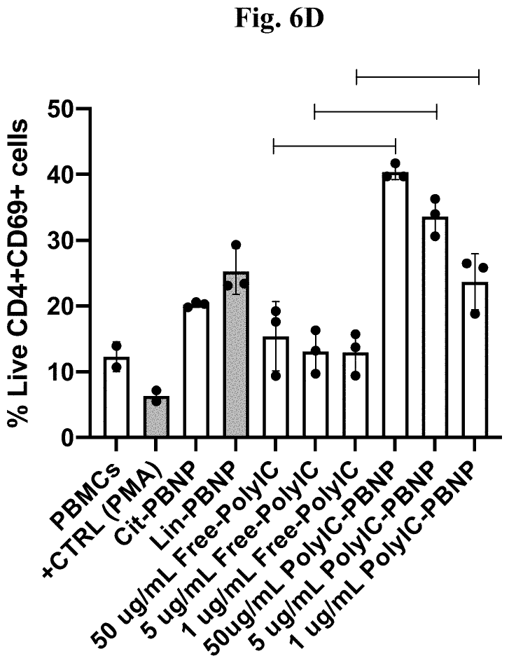

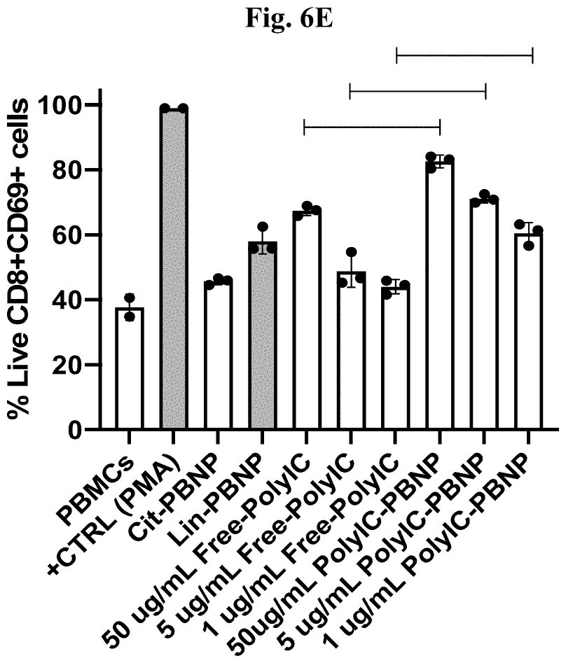

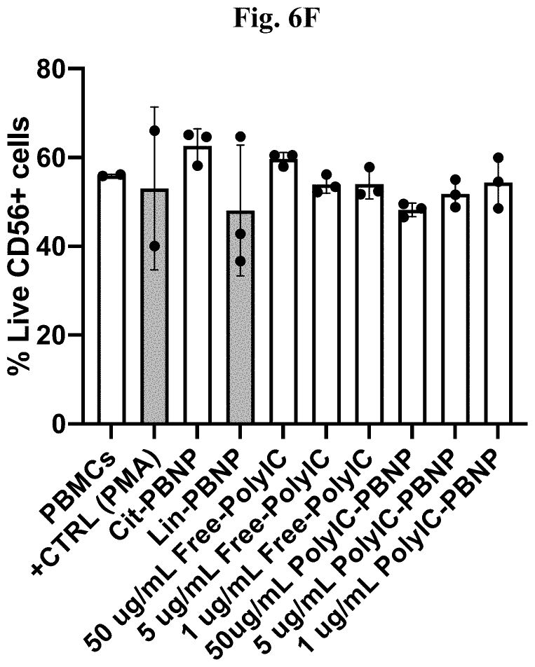

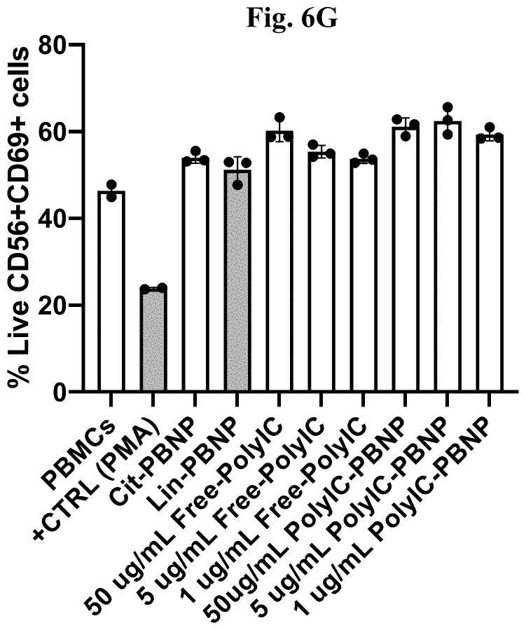

Example 4. Primary peripheral blood mononuclear cells (PBMCs) were isolated from blood collected from healthy human donors. PBMCs were incubated with increasing concentrations of Poly-PBNP (50, 5 and 1 μg/mL of attached PolyIC) and same concentrations of Free-Poly I:C overnight. PMA (Phorbol 12-myristate 13-acetate), a known immune-stimulator, was used as positive control herein. Following this treatment, the number of viable cells was assessed. FIG. 6A shows that PBMCs did not demonstrate toxicity to the varying concentrations of Poly-PBNP (50, 5 and 1 μg/mL of attached Poly I:C) and same concentrations of Free-Poly I:C.

Treated PBMCs were subjected to flow cytometry to examine ratios of immune cells in response to Poly-PBNP and Free-Poly I:C treatments. FIG. 6B shows that the ratio of T cells (CD3+) to other mononuclear cells (CD3−) did not change at the studied concentration of PolyIC-PBNP compared to untreated PBMCs (PBMCs). FIG. 6C shows that the ratio of T helper cells (CD4+) and cytotoxic T cells (CD8+) did not vary at the investigated PolyIC-PBNP concentrations compared to untreated PBMCs. The immune cell activation marker (CD69) was upregulated in both CD4+ T cells (FIG. 6D) and CD8+ T cells (FIG. 6E) for all concentrations of PolyIC-PBNP compared to their respective concentrations of Free-PolyIC. There was no change in the amount of NK cells (CD56+ cells) for any of the treatment groups studied, compared to untreated PBMCs (FIG. 6F). Additionally, treatment with Free-PolyIC and PolyIC-PBNP showed no changes in NK cells activation (CD56+CD69+) compared to untreated PBMCs (FIG. 6G).

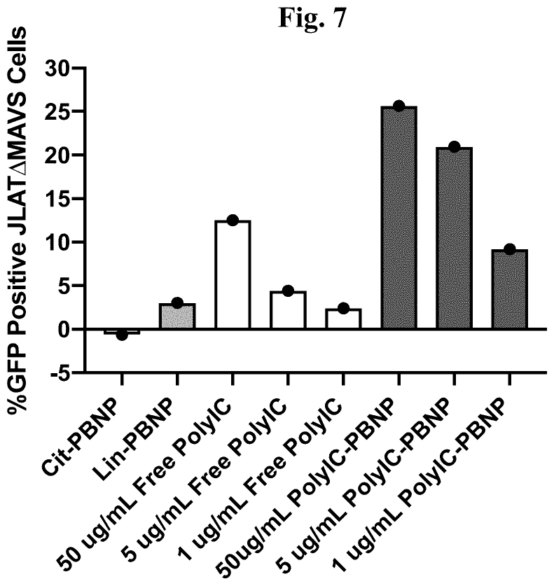

Example 5. Primary peripheral blood mononuclear cells (PBMCs) were isolated from blood collected from healthy human donors. PBMCs were incubated with increasing concentrations of Poly-PBNP (50, 5 and 1 μg/mL of attached PolyIC) and same concentrations of Free-Poly I:C overnight. Following overnight incubation, the supernatant was harvested from the PBMCs. The supernatants were then added to J-LAT cells lacking MAVS (J-LATAMAVS cells). Reactivation of the HIV virus was measured as a percentage of GFP expression. FIG. 7 shows that there was a clear increase in reactivation of JLATΔMAVS cells using PolyIC-PBNP compared to respective concentrations of Free Poly I:C.

1. A biofunctionalized nanocomposite, comprising:
(a) a core comprising a nanoparticle formed of Prussian blue materials;
(b) a shell obtained by partially or completely encapsulating the Prussian blue core with at least one biocompatible coating comprising citrate; and
(c) at least one biomolecule attached to, or absorbed to, the biocompatible coating.

2. The biofunctionalized nanocomposite of claim 1, wherein the Prussian blue materials are iron hexacyanoferrate (II) compounds.

3. The biofunctionalized nanocomposite of claim 1, wherein the Prussian blue materials are represented by general formula (I):
AₓB_{w}M₄[M′(CN)₆]_{z}·nH₂O (I),
wherein
x is from 0, 0.1 or about 0.1 to about 1;
w is from 0, 0.1 or about 0.1 to about 1;
z is from about 0.1 to about 4;
n is from about 0.1 to about 24;
A represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof,
B represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof,
M represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof, and
M′ represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof.

4. The biofunctionalized nanocomposite of claim 1, further comprising a second biocompatible coating comprising dextran, chitosan, silica, polyethylene glycol (PEG), avidin, a protein, a nucleic acid, a carbohydrate, a lipid, neutravidin, streptavidin, gelatin, collagen, fibronectin, albumin, a serum protein, a lysozyme, a phospholipid, a polyvinyl pyrrolidone (PVP), a polyvinyl alcohol, polyethylene glycol diacrylate, polyethylenimine (PEI), or any combination thereof.

5. The biofunctionalized nanocomposite of claim 4, wherein the second biocompatible coating comprises polyethylenimine (PEI).

6. (canceled)

7. The biofunctionalized nanocomposite of claim 5, wherein the biocompatible coating comprising citrate comprises at least one layer of citrate, and/or wherein the biocompatible coating comprising PEI comprises at least one layer of PEI.

8. The biofunctionalized nanocomposite of claim 1, wherein the at least one biomolecule attached to, or absorbed to, the biocompatible coating comprises an antibody, a peptide, a protein, an enzyme, an amino acid, a nucleic acid, a carbohydrate, a fat, an aptamer, a small molecule, a synthetic molecule, or any combination thereof.

9. The biofunctionalized nanocomposite of claim 1, wherein the at least one biomolecule attached to, or absorbed to, the biocompatible coating comprises at least one latency reversing agent.

10. The biofunctionalized nanocomposite of claim 9, wherein the at least one latency reversing agent comprises prostratin, bryostratin, ingenol, TNF-alpha, IL-15, an IL-15 superagonist, or any combination thereof.

11. The biofunctionalized nanocomposite of claim 9, wherein the at least one latency reversing agent comprises at least one pathogen recognition receptor agonist, and wherein the at least one pathogen recognition receptor agonist comprises a toll-like receptor activator, a RIG-1-like receptors activator, a cytosolic DNA sensors activator, a cyclic dinucleotide, or any combination thereof.

12. (canceled)

13. The biofunctionalized nanocomposite of claim 9, wherein the at least one latency reversing agent comprises at least one toll-like receptor (TLR) agonist, and wherein the at least one toll-like receptor agonist comprises a TLR-2 agonist, a TLR-3 agonist, a TLR-4 agonist, a TLR-5 agonist, a TLR-7 agonist, a TLR-7/8 agonist, a TLR-9 agonist, or any combination thereof.

14. (canceled)

15. The biofunctionalized nanocomposite of claim 9, wherein the at least one latency reversing agent comprises high molecular weight polyinosine-polycytidylic acid (poly I:C), low molecular weight poly I:C, Flagellin, GS-9620, R-848, CpG-ODNs, 5′ppp-dsRNA, 3p-hpRNA, Poly(I:C)/LyoVec complexes, Poly(dA:dT)/LyoVec complexes, 2′3′-cGAMP, 3′3′-cGAMP, c-di-AMP, c-di-GMP, cAIMP (CL592), cAIMP Difluor (CL614), cAIM(PS)2 Difluor (Rp/Sp) (CL656), 2′2′-cGAMP, 2′3′-cGAM(PS)2 (Rp/Sp), 3′3′-cGAMP Fluorinated, c-di-AMP Fluorinated, 2′3′-c-di-AMP, 2′3′-c-di-AM(P S)2 (Rp,Rp), c-di-GMP Fluorinated, 2′3′-c-di-GMP, c-di-IMP, dsDNA-EC, G3-YSD, HSV-60, ISD, ODN TTAGGG (A151), Poly(dA:dT), Poly(dG:dC), VACV-70, or any combination thereof.

16. The biofunctionalized nanocomposite of claim 9, wherein the at least one latency reversing agent comprises a high molecular weight polyinosine-polycytidylic acid (poly I:C) or a low molecular weight poly I:C.

17. The biofunctionalized nanocomposite of claim 1, wherein the at least one biomolecule attached to, or absorbed to, the biocompatible coating comprises at least one broadly neutralizing antibody (bnAB) against HIV, and wherein the at least one bnAB comprises 3BNC117, VRC01, VRC02, or 10-1074.

18. (canceled)

19. (canceled)

20. (canceled)

21. (canceled)

22. A method of treating HIV in a subject in need thereof, the method comprising: administering to a subject having or suspected of having HIV a therapeutically effective amount of a pharmaceutical composition comprising the biofunctionalized nanocomposite of claim 1, wherein, following administration, latent HIV reservoirs in the subject's cells are reduced and/or eradicated.

23. The method of claim 22, further comprising administering to the subject at least one latency reversing agent, at least one broadly neutralizing antibody against HIV, at least one anti-retroviral therapeutic, or any combination thereof.

24. (canceled)

25. (canceled)

26. (canceled)

27. (canceled)

28. The method of claim 23, wherein the at least one anti-retroviral therapeutic comprises enfuvirtide, zidovudine, abacavir, lamivudine, emtricitabine, tenfovir, nevirpine, efavirenz, etravirine, rilpivirine, raltegravir, elvitegravir, dolutegravir, lopinavir, indinavir, nelfinavir, amprenavir, ritonavir, darunavir, atazanavir, bevirimat, vivecon, stavudine, didanosine, delavirdine, nevirapine, fosamprenavir, saquinavir, tipranavir, maraviroc, or any combination thereof.

29. The method of claim 23, wherein the at least one anti-retroviral therapeutic comprises at least one nucleoside/nucleotide reverse transcriptase inhibitor (NRTI), at least one non-nucleoside reverse transcriptase inhibitor (NNRTI), at least one protease inhibitor, at least one fusion or entry inhibitor, at least one integrase inhibitor, or any combination thereof.

30. (canceled)

31. (canceled)

32. (canceled)

33. (canceled)

34. (canceled)

35. The method of claim 22, wherein the biofunctionalized nanocomposite further comprises a second biocompatible coating, wherein the second biocompatible coating comprises polyethylenimine (PEI).

36. The method of claim 35, wherein the at least one biomolecule attached to, or absorbed to, the biocompatible coating comprises at least one latency reversing agent, and wherein the at least one latency reversing agent comprises a high molecular weight polyinosine-polycytidylic acid (poly I:C) or low molecular weight poly I:C.
